(19) United States
(12) Patent Application Publication
(10) Pub. No.: US 2026/0286393 A1
(43) Pub. Date: Sep. 24, 2026

(54) USE OF ONCOLYTIC VIRUSES FOR THE TREATMENT OF CANCER

(71) Applicant: AMGEN INC., Thousand Oaks, CA (US)

(72) Inventors: Jason James DEVOSS, Burlingame, CA (US); Walter Hans MEISEN, Foster City, CA (US); Christine Elaine TINBERG, San Francisco, CA (US); Keegan S. COOKE, Ventura, CA (US); Achim Klaus MOESTA, Woodside, CA (US)

(73) Assignee: AMGEN INC., Thousand Oaks, CA (US)

(21) Appl. No.: 19/573,124

(22) Filed: Mar. 20, 2026

Related U.S. Application Data
(63) Continuation of application No. 17/435,768, filed on Mar. 3, 2020.
(60) Provisional application No. 62/813,961, filed on Mar. 5, 2019.

(51) Int. Cl. C12N 15/86; A61K 38/17; A61K 38/20; A61P 35/00; C07K 14/54; C07K 14/71; C12N 7/00

(52) U.S. Cl. CPC C12N 15/86; A61K 38/179; A61K 38/208; A61P 35/00; C07K 14/5434; C07K 14/71; C12N 7/00; C07K 2319/00; C12N 2710/16632; C12N 2710/16643; C12N 2710/16671; C12N 2830/50

(57) ABSTRACT

The present invention relates to the use of oncolytic viruses (e.g., modified HSV-1 viruses) for the treatment of various types of cancer. In addition, the present invention relates to compositions and kits relating to such uses of oncolytic viruses.

### CROSS REFERENCE TO RELATED APPLICATIONS

This application claims priority to and the benefit of U.S. Provisional Application No. 62/813,961 filed Mar. 5, 2019, which is incorporated by reference herein in its entirety.

### REFERENCE TO THE SEQUENCE LISTING

This application contains a Sequence Listing in computer-readable form. The Sequence Listing is provided as a text file entitled A-2353-WO-PCT_SeqListing_ST25.txt, created Jan. 10, 2020, which is 37,667 bytes in size. The information in the electronic format of the Sequence Listing is incorporated herein by reference in its entirety.

### BACKGROUND OF THE INVENTION

The recent advances in the treatment of many forms of cancer have greatly improved the rate of survival for both men and women for the most common types of cancer such as lung cancer, colon cancer, breast cancer, and prostate cancer. The advent of checkpoint inhibitors, which have been successful at directing a patient's immune system to attack certain forms of cancer, has greatly improved patient survival for certain cancers. For example, checkpoint inhibitors, such as ipilimumab (an anti-CTLA-4 antibody), pembrolizumab and nivolumab (anti-PD-1 antibodies), and atezolizumab (an anti-PD-L1 antibody) have demonstrated efficacy in a variety of tumor types. See, Grosso et al., Cancer Immun., 13:5 (2013); Pardoll, Nat Rev Cancer, 12:252-264 (2012); and Chen et al., Immunity, 39:1-10 (2013).

Oncolytic viruses have also demonstrated clinical efficacy in the treatment of certain forms of cancer. Oncolytic viruses are typically genetically engineered to preferentially replicate in cancer cells (over healthy cells) and to include “payloads” which can be used to enhance the antitumor response. Such genetic engineering initially focused on the use of replication-incompetent viruses in a bid to prevent virus-induced damage to non-tumor cells. More recently, genetic engineering of oncolytic viruses has focused on the generation of “replication-conditional” viruses to avoid systemic infection while allowing the virus to spread to other tumor cells.

Currently, the only approved oncolytic virus-based drug in the U.S. and Europe is talimogene laherparepvec (IMLYGIC®). Talimogene laherparepvec is an HSV-1 derived from the clinical strain JS1 (deposited at the European collection of cell cultures (ECAAC) under accession number 01010209). In talimogene laherparepvec, the HSV-1 viral genes encoding ICP34.5 and ICP47 have been functionally deleted. Functional deletion of ICP47 leads to earlier expression of US11, a gene that promotes virus growth in tumor cells without decreasing tumor selectivity. In addition, the coding sequence for human GM-CSF has been inserted into the viral genome at the former ICP34.5 gene sites. See, Liu et al., Gene Ther., 10:292-303, 2003.

Therapeutic combinations of oncolytic viruses and checkpoint inhibitors have been explored. For example, combinations of talimogene laherparepvec and immunotherapies (e.g., ipilimumab and pembrolizumab) are currently being explored in clinical trials in melanoma (NCT01740297 and NCT02263508) and squamous cell carcinoma of the head and neck (NCT02626000).

Although oncolytic viruses have demonstrated great promise in the treatment of cancer, there remains a need to develop oncolytic viruses that not only limit their replication and lytic damage to cancer cells, but are also able to aid in the mounting and maintenance of a robust systemic anti-tumor immune response.

The present invention addresses these and other needs.

### SUMMARY OF THE INVENTION

The present invention relates to oncolytic viruses comprising a nucleic acid encoding a heterologous dendritic cell growth factor and a nucleic acid encoding a first heterologous cytokine. The heterologous dendritic cell growth factor and first heterologous cytokine may be linked by a polycistronic linker element. In some embodiments, the polycistronic linker element is porcine tescho virus 2a (P2A) or internal ribosomal entry site (IRES). The oncolytic virus may be a herpes simplex virus, such as a herpes simplex-1 virus. In a particular embodiment, the oncolytic virus is derived from the HSV-1 strain JS1.

The oncolytic virus may be further modified so that it lacks a functional ICP 34.5 gene and lacks a functional ICP 47 gene.

In addition, the oncolytic virus may further comprise a promoter wherein the nucleic acid sequences encoding the dendritic cell growth factor and first cytokine are both under the control of the same promoter. In other embodiments, the oncolytic virus may comprise a first promoter, wherein the nucleic acid sequence encoding the dendritic cell growth factor is under the control of the first promoter; and a second promoter, wherein the nucleic acid sequence encoding the first cytokine is under the control of the second promoter.

The first heterologous cytokine may be an interleukin, such as interleukin-12 (IL12). The heterologous dendritic cell growth factor may be a second cytokine, such as Fms-related tyrosine kinase 3 ligand (FLT3L).

In a particular embodiment, the oncolytic virus of the present invention comprises an HSV-1 that lacks a functional ICP34.5 encoding gene and lacks a functional ICP47 encoding gene, comprises a nucleic acid encoding FLT3L, and further comprises a nucleic acid encoding IL12. In some embodiments, the nucleic acid encoding IL12 and the nucleic acid encoding FLT3L are present in the former site of the ICP34.5 encoding gene. In one embodiment, the nucleic acid encoding IL12 and the nucleic acid encoding FLT3L are linked via P2A.

The nucleic acids encoding IL12, FLT3L, and P2A may be present as: [Flt3L]-[P2A]-[IL12], wherein the [Flt3L]-[P2A]-[IL12] construct is under the control of a single promoter, and the construct is present in the former site of the ICP34.5 encoding gene. Suitable promoters include: cytomegalovirus (CMV), rous sarcoma virus (RSV), human elongation factor 1α promoter (EF1a), simian virus 40 early promoter (SV40), phosphoglycerate kinase 1 promoter (PGK), ubiquitin C promoter (UBC), and murine stem cell virus (MSCV). In a particular embodiment, the promoter is CMV.

The oncolytic viruses of the present invention may comprise a bovine growth hormone polyadenylation signal sequence (BGHpA). The oncolytic viruses of the present invention may also comprise a nucleic acid that enhances mammalian translation. In some embodiments, the nucleic acid that enhances mammalian translation is a Kozak sequence or a consensus Kozak sequence. In a particular embodiment, the consensus Kozak sequence is recited in SEQ ID NO: 20.

In one embodiment, the oncolytic virus comprises a nucleic acid, or nucleic acids (also referred to as a construct or an expression cassette), encoding [CMV]-[Kozak]-[Flt3L]-[P2A]-[IL12]-[BGHpA]. In another embodiment, IL12 is present as [P40 subunit]-[GGGGS]-[P35 subunit]. In another embodiment, the signal peptide in the IL12 P35 subunit is absent. In another embodiment, the oncolytic virus comprises a nucleic acid, or nucleic acids, encoding [CMV]-[Kozak]-[Flt3L]-[P2A]-[IL12(p40-GGGGS-No SP-p35)]-[BGHpA]. In yet another embodiment, the construct is present in the former site of the ICP34.5 encoding gene. The orientation of the construct within the former site of the ICP34.5 encoding gene used to generate HSV-1/ICP34.5⁻/ICP47⁻/FLT3L/IL12 is displayed in FIG. 9, though multiple orientations of the expression cassette within the former site of the ICP34.5 encoding gene could be generated/utilized.

In some embodiments, the oncolytic virus comprises a FLT3L sequence comprising SEQ ID NO: 1 and an IL12 sequence comprising SEQ ID NO: 7.

In some embodiments, the oncolytic virus comprises a CMV promotor comprising SEQ ID NO: 24, a Kozak sequence comprising SEQ ID NO: 20, a FLT3L sequence comprising SEQ ID NO: 1, a P2A sequence (GSG-P2A) SEQ ID NO: 17, an IL12 sequence comprising SEQ ID NO: 7, and a BGHpA sequence comprising SEQ ID NO: 21.

The present invention also includes methods of treating cancer using the oncolytic virus of the present invention. In addition, the present invention includes a therapeutically effective amount of the oncolytic virus for use in treating cancer.

The present invention also includes pharmaceutical compositions for use in treating cancer. The pharmaceutical compositions may further comprise a checkpoint inhibitor.

In some embodiments, the present invention includes a kit comprising an oncolytic virus of the present invention.

### BRIEF DESCRIPTION OF THE DRAWINGS

FIG. 1. FIG. 1 shows the in-silico modeling of linkers evaluated for the fusion of the IL12p35 and IL12p40 chains to create a single chain cytokine product.

FIG. 2. FIG. 2 shows the energy conformation modeling for linkers evaluated for the fusion of IL12p35 and IL12p40 chains.

FIG. 3. FIG. 3 shows the engineering of the IL12 fusion protein to optimize expression including assessment of the orientation of chains, the placement of signal peptides, and the linker used.

FIG. 4. FIG. 4 shows the expression of FLT3L and single chain IL12 when expressed with a porcine 2A virus (P2A) sequence or an internal ribosomal entry site (IRES) sequence.

FIG. 5. FIG. 5 shows the effect of KOZAK sequence incorporation into the DNA construct on the level of cytokine product produced.

FIG. 6. FIG. 6 shows structural impact of P2A amino acid addition to the activity and receptor binding of FLT3L to its cognate receptor, FLT3.

FIG. 7. FIG. 7 shows the activity of recombinant human IL12 (A) and the single chain IL12 produced by the FLT3L-P2A-IL12 construct (B) in an in vitro reporter assay.

FIG. 8. FIG. 8 shows the activity of recombinant human FLT3L (A) and FLT3L produced by the FLT3L-P2A-IL12 construct (B) in an in vitro cellular proliferation assay.

FIG. 9. FIG. 9 shows the homologous recombination approach to generate the engineered virus containing the FLT3-IL12 sequence inserted into the two 34.5 loci of the HSV1 genome.

FIG. 10. FIG. 10 shows the in vitro replication capacity of the HSV-1/ICP34.5⁻/ICP47⁻/FLT3L/IL12 virus in VERO (A) and A375 (B) cell lines.

FIG. 11. FIG. 11 shows the in vitro infection and lytic capacity of the HSV-1/ICP34.5⁻/ICP47⁻/FLT3L/IL12 virus in mouse CT26 cells (A) and human HT-29 (B), SK-MEL-5 (C), FADU (D) and BxPC-3 cell lines (E).

FIG. 12. FIG. 12 shows the expression of FLT3L and IL12 from the HSV-1/ICP34.5⁻/ICP47⁻/FLT3L/IL12 virus in infected human VERO, SK-MEL-5, and A375 cells.

FIG. 13. FIG. 13 shows the activity of IL12 when expressed by human SK-MEL-5 (A) or A375 (B) cells infected with HSV-1/ICP34.5⁻/ICP47⁻/FLT3L/IL12 virus in vitro.

FIG. 14. FIG. 14 shows that activity of FLT3L when expressed by human SK-MEL-5 (A) or VERO (B) cells infected with HSV-1/ICP34.5⁻/ICP47⁻/FLT3L/IL12 virus in vitro.

FIG. 15. FIG. 15 shows the in vivo expression of mouse FLT3L and IL12 from A20 tumor cells implanted on BALB/c animals and injected intratumorally with 1e6 PFU/animal of HSV-1/ICP34.5⁻/ICP47⁻/mFLT3L/mIL12.

FIG. 16. FIG. 16 shows the in vivo expression of mouse FLT3L and IL12 from B16F10 tumor cells implanted on C57BL6 animals and injected intratumorally with 5e6 PFU/animal of HSV-1/ICP34.5⁻/ICP47⁻/mFLT3L/mIL12.

FIG. 17. FIG. 17 shows anti-tumor T cell responses that occur as a result of injection with an HSV-1/ICP34.5⁻/ICP47⁻/mGMCSF or HSV-1/ICP34.5⁻/ICP47⁻/mFLT3L/mIL12 virus.

FIG. 18. FIG. 18 shows the anti-tumor efficacy of HSV-1/ICP34.5⁻/ICP47⁻/mGMCSF and HSV-1/ICP34.5⁻/ICP47⁻/mFLT3L/mIL12 in a bilateral mouse syngeneic B cell lymphoma (A20 cell line) tumor model where virus was delivered intratumorally to only one of the tumors (right flank) and the other tumor was left untreated (left flank).

FIG. 19. FIG. 19 shows the anti-tumor efficacy of HSV-1/ICP34.5⁻/ICP47⁻/mGMCSF and HSV-1/ICP34.5⁻/ICP47⁻/mFLT3L/mIL12 in a bilateral mouse syngeneic neuroblastoma (Neuro2A cell line) tumor model where virus was delivered intratumorally to only one of the tumors (right flank) and the other tumor was left untreated (left flank).

FIG. 20. FIG. 20 shows the anti-tumor efficacy of HSV-1/ICP34.5⁻/ICP47⁻/mGMCSF and HSV-1/ICP34.5⁻/ICP47⁻/mFLT3L/mIL12 in a bilateral mouse syngeneic colorectal (CT26 cell line) tumor model where virus was delivered intratumorally to only one of the tumors (right flank) and the other tumor was left untreated (left flank).

FIG. 21. FIG. 21 shows the anti-tumor efficacy of HSV-1/ICP34.5⁻/ICP47⁻/mFLT3L/mIL12 in combination with checkpoint blockade (anti-PD1 mAb) in a bilateral mouse syngeneic colorectal (MC38 cell line) tumor model where virus was delivered intratumorally to only one of the tumors (right flank) and the other tumor was left untreated (left flank).

FIG. 22. FIG. 22 shows the cytokine/payload production of HSV-1/ICP34.5⁻/ICP47⁻/mFLT3L/mIL12 in a single mouse syngeneic colorectal (CT26 cell line) tumor model where virus was delivered intratumorally to the tumor (right flank).

FIG. 23. FIG. 23 shows the anti-tumor response (as measured by ELISpot) generated by the injection of HSV-1/ICP34.5⁻/ICP47⁻/mFLT3L/mIL12 alone or in combination with an anti-PD1 antibody in a bilateral mouse syngeneic colorectal (MC38 cell line) tumor model. Lines underneath the X-axis represent the results of a statistical analysis (two tailed students T test) between the groups indicated at the start and end of the line. P values are denoted as follows: * is p≤0.05; ** is p≤0.01, * is p≤0.001, **** is p≤0.0001

FIG. 24. FIG. 24 shows the anti-tumor efficacy of HSV-1/ICP34.5⁻/ICP47⁻/mFLT3L/mIL12 in combination with an anti-4-1BB agonist antibody in a bilateral mouse syngeneic colorectal (MC38 cell line) tumor model where virus was delivered intratumorally to only one of the tumors (right flank) and the other tumor was left untreated (left flank).

### DETAILED DESCRIPTION

The section headings used herein are for organizational purposes only and are not to be construed as limiting the subject matter described. All references cited within the body of this specification are expressly incorporated by reference in their entirety.

Unless otherwise defined herein, scientific and technical terms used in connection with the present application have the meanings that are commonly understood by those of ordinary skill in the art. Further, unless otherwise required by context, singular terms shall include pluralities and plural terms shall include the singular.

Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present application are generally performed according to conventional methods well known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001), Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients.

It should be understood that this invention is not limited to the particular methodology, protocols, and reagents, etc., described herein and as such may vary. The terminology used herein is for the purpose of describing particular embodiments only and is not intended to limit the scope of the disclosed, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

All embodiments narrower in scope in any way than the variations defined by specific paragraphs herein are to be considered included in this disclosure. For example, certain aspects are described as a genus, and it should be understood that every member of a genus can be, individually, an embodiment. Also, aspects described as a genus or selecting a member of a genus should be understood to embrace combinations of two or more members of the genus. It should also be understood that while various embodiments in the specification are presented using “comprising” language, under various circumstances, a related embodiment may also be described using “consisting of” or “consisting essentially of” language.

### Definitions

The term “functionally deleted” when referring to a gene means that the gene is modified (e.g., by partially or completely deleting, replacing, rearranging, or otherwise altering the gene) such that a functional protein can no longer be expressed from that gene. In the context of a herpes simplex virus (such as an oncolytic virus), a gene is “functionally deleted” when the viral gene is modified in the herpes simplex genome such that a functional viral protein can no longer be expressed from that gene by the herpes simplex virus.

The term “heterologous” when referring to the nucleic acid (or the protein encoded by the nucleic acid) present in the viral genome refers to a nucleic acid that is not naturally present in the virus (or a protein that is not naturally produced by the virus). For example, a nucleic acid encoding human IL12 or a nucleic acid encoding human FLT3L would be “heterologous” with respect to HSV-1.

The term “oncolytic virus” refers to a virus that, naturally or as a result of modification, preferentially infects and kills cancer cells versus non-cancer cells.

As used herein, the terms “patient” or “subject” are used interchangeably and mean a mammal, including, but not limited to, a human or non-human mammal, such as a bovine, equine, canine, ovine, or feline. Preferably, the patient is a human.

The term “HSV1/ICP34.5⁻/ICP47⁻/FLT3L/IL12” refers to a modified HSV-1 derived from strain JS1, wherein the HSV-1 lacks a functional ICP34.5 encoding gene, lacks a functional ICP47 encoding gene, comprises the following inserted into the former sites of the ICP 34.5 gene: [CMV]-[Kozak]-[Flt3L]-[P2A]-[IL12(p40-GGGGS-No SP-p35)]-[BGHpA].

### Oncolytic Viruses

Any virus can be used to generate the oncolytic virus of the present invention. Generally, the virus can be modified to, e.g., modulate its replication (e.g., to preferentially replicate in tumor cells versus healthy cells), its ability to be detected by the host's immune system, and to include exogenous nucleic acids.

In some embodiments, the oncolytic virus is a herpes simplex virus (HSV). In other embodiments, the oncolytic virus is a herpes simplex-1 virus (HSV-1). In yet other embodiments, the oncolytic virus is derived from JS1 (an HSV-1). JS1 as deposited at the European collection of cell cultures (ECAAC) under accession number 01010209.

In some embodiments, the oncolytic virus is an HSV-1 wherein the viral genes encoding ICP34.5 are functionally deleted. Functional deletion of ICP34.5, which acts as a virulence factor during HSV infection, limits replication in non-dividing cells and renders the virus non-pathogenic. The safety of ICP34.5-functionally deleted HSV has been shown in multiple clinical studies (MacKie et al, Lancet 357: 525-526, 2001; Markert et al, Gene Ther 7: 867-874, 2000; Rampling et al, Gene Ther 7:859-866, 2000; Sundaresan et al, J. Virol 74: 3822-3841, 2000; Hunter et al, J Virol August; 73(8): 6319-6326, 1999).

In other embodiments, the oncolytic virus is an HSV-1 wherein the viral gene encoding ICP47 (which blocks viral antigen presentation to major histocompatibility complex class I and II molecules) is functionally deleted. Functional deletion of ICP47 also leads to earlier expression of US11, a gene that promotes virus growth in tumor cells without decreasing tumor selectivity.

In some embodiments, the viral genes encoding ICP34.5 are deleted. In some embodiments, the viral genes encoding ICP47 are deleted. In some embodiments, both the viral genes encoding ICP34.5 and the viral gene encoding ICP47 are deleted. In some embodiments, both the viral genes encoding ICP34.5 and the viral gene encoding ICP47 are deleted, and the deletion of ICP47 leads to earlier expression of US11.

Herpes virus strains and how to make such strains are described in U.S. Pat. Nos. 5,824,318; 6,764,675; 6,770,274; 7,063,835; 7,223,593; 7,749,745; 7,744,899; 8,273,568; 8,420,071; 8,470,577; WIPO Publication Numbers: WO199600007; WO199639841; WO199907394; WO200054795; WO2006002394; WO201306795; Chinese Patent Numbers: CN128303, CN10230334 and CN 10230335; Varghese and Rabkin, (2002) Cancer Gene Therapy 9:967-97 and Cassady and Ness Parker, (2010) The Open Virology Journal 4:103-108, each of which is incorporated herein by reference.

The oncolytic viruses of the present invention are also modified so that they contain exogenous nucleic acid(s) encoding proteins. Such proteins were rationally selected to enhance the immunostimulatory capacity of the virus. Increasing the immunostimulatory capacity allows the oncolytic virus to elicit a more robust anti-tumor response. Thus, in one aspect, the oncolytic virus comprises a nucleic acid encoding a heterologous dendritic cell growth factor, a first heterologous cytokine, or both. FLT3L enhances the proliferation and survival of dendritic cells, especially the cDC1 subset, which is critical for the cross-presentation of tumor antigens to T cells. In addition, IL12 augments T helper type 1 (Th1) and cytotoxic T lymphocyte (CTL) function, resulting in maximal tumor killing activity. Without being bound by a theory, it is thought that the combination of these two sets of attributes would yield an oncolytic virus which is surprisingly capable of, e.g., inducing a systemic immune response to cancer cells.

In a particular embodiment, the oncolytic virus comprises a nucleic acid encoding a heterologous dendritic cell growth factor and a nucleic acid encoding a first heterologous cytokine (sometimes referred to as “payloads”). Examples of first heterologous cytokines include interleukin-2 (IL2), IL7, IL12, IL15, IL21, TNF, and other members of the interleukin family of cytokines and proteins capable of binding to receptors on immune cells and/or capable of augmenting T cell function or memory formation. In a particular embodiment, the first heterologous cytokine is IL12 (murine or human). The nucleic acid sequences encoding muIL12a and muIL12b are recited in SEQ ID NOs: 11 and 13, respectively. The nucleic acid sequences encoding huIL12a and huIL12b are recited in SEQ ID NOs: 3 and 5, respectively. The amino acid sequences of muIL12a and muIL12b are recited in SEQ ID NOs: 12 and 14, respectively. The amino acid sequences of huIL12a and huIlL2b are recited in SEQ ID NOs: 4 and 6, respectively.

In native form, IL12 is a heterodimeric cytokine comprising IL12A (p35 subunit) and IL12B (p40 subunit), wherein each subunit is encoded by a separate gene. Thus, in some embodiments, the oncolytic virus of the present invention comprises two heterologous nucleic acids: one encoding the IL12 p35 subunit, and the other encoding the IL12 p40 subunit. In other embodiments, the oncolytic virus of the present invention comprises a single chain IL12 variant. In such single chain IL12 variants, the p35 and p40 subunits can be directly fused to each other (i.e., without a linker) or can be joined to each other via a linker (either synthetic or peptide-based). Examples of suitable linkers include: elastin-based linkers (VPGVGVPGVGGS; nucleic acid sequence shown in SEQ ID NO: 22; amino acid sequence shown in SEQ ID NO: 23), G₄S, 2×(G₄S), 3×(G₄S), 4×(G₄S), 5×(G₄S), 6×(G₄S), 7×(G₄S), 8×(G₄S), 9×(G₄S), and 10×(G₄S). In some embodiments, the linker is VPGVGVPGVGGS, G₄S, 2×(G₄S), or 3×(G₄S). In a particular embodiment, the linker is G₄S.

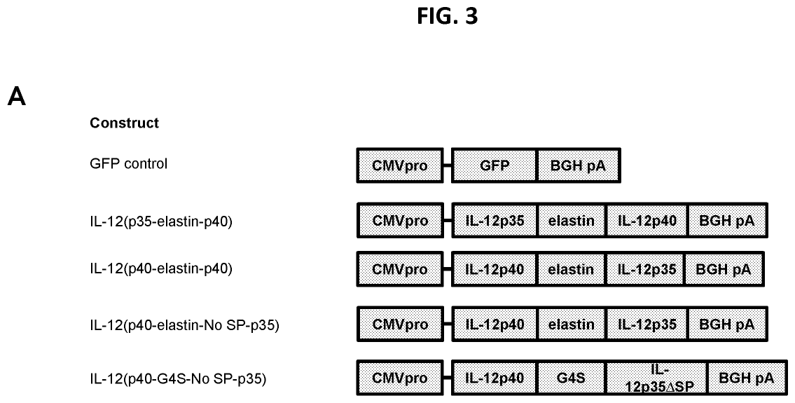

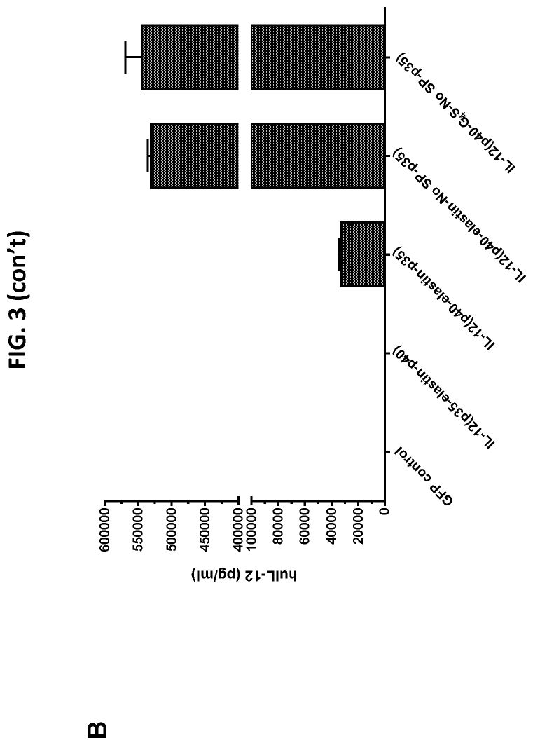

IL12 variants may contain or may exclude the signal peptides (one for each subunit) present in the native IL12 protein. In some embodiments, the IL12 variant contains none of, one of, or both of the signal peptides. In a specific embodiment, the IL12 variant contains a single signal peptide—e.g., [IL12(p40-GGGGS-No SP-p35)](nucleic acid sequence present in SEQ ID NO: 7; amino acid sequence present in SEQ ID NO: 8) where the p40 signal peptide is maintained and the p35 signal peptide is removed. See, FIG. 3.

Examples of heterologous dendritic cell growth factors include cytokines, C-type lectins, and CD40L. In some embodiments, the heterologous dendritic cell growth factor is a cytokine (i.e., a second cytokine) selected from the list comprising: Fms-related tyrosine kinase 3 ligand (FLT3L), GMCSF, TNFα, IL36γ, and IFN. In a particular embodiment, the heterologous dendritic cell growth factor is FLT3L. The nucleic acid sequence encoding muFLT3L is recited in SEQ ID NO: 9. The nucleic acid sequence encoding huFLT3L is recited in SEQ ID NO: 1. The amino acid sequence of muFLT3L is recited in SEQ ID NO: 10. The amino acid sequence of huFLT3L is recited in SEQ ID NO: 2.

In some embodiments, the oncolytic virus comprises nucleic acid(s) encoding FLT3L and IL12. In other embodiments, the oncolytic virus is an HSV-1 wherein the viral genes encoding ICP34.5 and the viral gene encoding ICP47 are deleted, and the oncolytic virus comprises nucleic acid(s) encoding FLT3L and IL12.

The exogenous nucleic acids may be under the control of the same promoter or different promoters. In a particular embodiment, the nucleic acid encoding the heterologous dendritic cell growth factor and the nucleic acid encoding a first heterologous cytokine are under the control of the same promoter. Using a single promoter (e.g., a CMV promoter) has the benefit of producing both the heterologous dendritic cell growth factor and the first heterologous cytokine in the same infected cell at the same rate and at the same time.

Examples of suitable promoters include: cytomegalovirus (CMV), rous sarcoma virus (RSV), human elongation factor 1α promoter (EF1a), simian virus 40 early promoter (SV40), phosphoglycerate kinase 1 promoter (PGK), ubiquitin C promoter (UBC), and murine stem cell virus (MSCV). In a particular embodiment, the promoter is CMV (nucleic acid sequence shown in SEQ ID NO: 24).

When under control of the same promoter, the nucleic acids encoding the payloads may be linked by additional nucleic acid which, e.g., allows polycistronic translation (polycistronic linker elements). Examples of suitable polycistronic linker elements include: ribosomal entry sites (e.g., internal ribosomal entry sites (IRES) (SEQ ID NO: 19)), 2A sequences (e.g., porcine tescho virus 2a (GSG-P2A; nucleic acid sequence recited in SEQ ID NO: 17; amino acid sequence recited in SEQ ID NO: 18), thosea asigna virus 2A (T2A), foot and mouth disease virus 2A (F2A), and equine rhinitis A virus (E2A)). Such sequences can be used to link the two nucleic acids in any orientation. For example, the nucleic acids in the viral genome may be oriented as such: [heterologous dendritic cell growth factor]-[P2A]-[first heterologous cytokine] or [first heterologous cytokine]-[P2A]-[heterologous dendritic cell growth factor].

It has been observed that the use of IRES leads to diminished production of the second nucleic acid 3′ of the IRES in the construct. For example, production of FLT3L in the [IL12]-[IRES]-[FLT3L] construct was decreased while production of IL12 in the [FLT3L]-[IRES]-[IL12] was decreased. See, Example 4. Accordingly, in one embodiment, the polycistronic linker element is 2A. In a specific embodiment, the polycistronic linker element is P2A.

The oncolytic viruses of the present invention can also contain sequences that enhance translation (e.g., mammalian translation) of exogenous nucleic acids. For example, KOZAK sequences are known to enhance mammalian translation. Thus, in some embodiments, the oncolytic virus comprises a Kozak sequence. In one embodiment the Kozak sequences is a consensus Kozak sequence (SEQ ID NO: 20).

The oncolytic viruses of the present invention may also contain sequences that enhance the stability of the virally expressed mRNAs. Examples of such sequences include bovine growth hormone polyadenylation signal sequence (BGHpA) and rabbit beta globin (RBGpA), SV40 polyA, and hGH polyA. In a specific embodiment, the sequence is BGHpA (SEQ ID NO: 21).

Other oncolytic viruses that may be modified as described herein include RP1 (HSV-1/ICP34.5⁻/ICP47⁻/GM-CSF/GALV-GP R(−); RP2 (HSV-1/ICP34.5⁻/ICP47⁻/GM-CSF/GALV-GP R(−)/anti-CTLA-4 binder; and RP3 (HSV-1/ICP34.5⁻/ICP47⁻/GM-CSF/GALV-GP R(−)/anti-CTLA-4 binder/co-stimulatory ligands (e.g., CD40L, 4-1BBL, GITRL, OX40L, ICOSL)). In such oncolytic viruses, GALV (gibbon ape leukemia virus) has been modified with a specific deletion of the R-peptide, resulting in GALV-GP R(−). Such oncolytic virsues are discussed in WO2017118864, WO2017118865, WO2017118866, WO2017118867, and WO2018127713A1, each of which is incorporated by reference in its entirety. Additional examples of oncolytic viruses that may be modified as described herein include NSC-733972, HF-10, BV-2711, JX-594, Myb34.5, AE-618, Brainwel™, and Heapwel™, Cavatak® (coxsackievirus, CVA21), HF-10, Seprehvir®, Reolysin®, enadenotucirev, ONCR-177, and those described in U.S. Pat. No. 10,105,404, WO2018006005, WO2018026872A1, and WO2017181420, each of which is incorporated by reference in its entirety.

Further examples of oncolytic viruses that may be modified as described herein include:

G207, an oncolytic HSV-1 derived from wild-type HSV-1 strain F having deletions in both copies of the major determinant of HSV neurovirulence, the ICP 34.5 gene, and an inactivating insertion of the E. coli lacZ gene in UL39, which encodes the infected-cell protein 6 (ICP6), see Mineta et al. (1995) Nat Med. 1:938-943.

OrienX010, a herpes simplex virus with deletion of both copies of γ34.5 and the ICP47 genes as well as an interruption of the ICP6 gene and insertion of the human GM-CSF gene, see Liu et al., (2013) World Journal of Gastroenterology 19(31):5138-5143.

NV1020, a herpes simples virus with the joint region of the long (L) and short (S) regions is deleted, including one copy of ICP34.5, UL24, and UL56.34,35. The deleted region was replaced with a fragment of HSV-2 US DNA (US2, US3 (PK), gJ, and gG), see Todo, et al. (2001) Proc Natl Acad Sci USA. 98:6396-6401.

M032, a herpes simplex virus with deletion of both copies of the ICP34.5 genes and insertion of interleukin 12, see Cassady and Ness Parker, (2010) The Open Virology Journal 4:103-108.

ImmunoVEX HSV2, is a herpes simplex virus (HSV-2) having functional deletions of the genes encoding vhs, ICP47, ICP34.5, UL43 and US5.

OncoVEX^{GALV/CD}, is also derived from HSV-1 strain JS1 with the genes encoding ICP34.5 and ICP47 having been functionally deleted and the gene encoding cytosine deaminase and gibbon ape leukaemia fusogenic glycoprotein inserted into the viral genome in place of the ICP34.5 genes.

In a particular embodiment, the oncolytic virus of the present invention is HSV1/ICP34.5⁻/ICP47⁻/FLT3L/IL12. In another embodiment, the oncolytic virus of the present invention is HSV1/ICP34.5⁻/ICP47⁻/FLT3L/IL12, wherein said virus is derived from HSV-1 strain JS1 deposited at the European collection of cell cultures (ECAAC) under accession number 01010209.

Combinations with Other Agents

The oncolytic viruses of the present invention can be used as single agents for the treatment of diseases such as cancer. Oncolytic viruses have generally been found to be safe with a favorable safety profile. Thus, the oncolytic viruses of the present invention can be used in combination with other agents without a significant negative contribution to the safety profile.

The oncolytic viruses of the present invention (e.g., HSV1/ICP34.5⁻/ICP47⁻/FLT3L/IL12) may be used in combination with immune checkpoint inhibitors, immune cytokines, agonists of co-stimulatory molecules, targeted therapies, as well as standard of care therapies. For example, the oncolytic viruses of the present invention (e.g., HSV1/ICP34.5⁻/ICP47⁻/FLT3L/IL12) may be used in combination with targeted cancer therapies (e g., MEK inhibitors such as cobimetinib, trametinib, and binimetinib) and/or cytokines (e.g., pegylated IL2 (e.g., bempegaldesleukin) or pegylated IL10 (e.g., pegilodecakin)).

### Checkpoint Inhibitors

Immune checkpoints are proteins which regulate some types of immune system cells, such as T cells (which play a central role in cell-mediated immunity). Although immune checkpoints aid in keeping immune responses in check, they can also keep T cells from killing cancer cells. Immune checkpoint inhibitors (or simply “checkpoint inhibitors”) can block immune checkpoint protein activity, releasing the “brakes” on the immune system, and allowing T cells to better kill cancer cells.

As used herein, the term “immune checkpoint inhibitor” or “checkpoint inhibitor” refers to molecules that totally or partially reduce, inhibit, interfere with or modulate one or more checkpoint proteins. Checkpoint proteins regulate T-cell activation or function. Numerous checkpoint proteins are known, such as CTLA-4 and its ligands CD80 and CD86; and PD-1 with its ligands PD-L1 and PD-L2 (Pardoll, Nature Reviews Cancer 12: 252-264, 2012). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or can be derived from antibodies.

Checkpoint inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PD-L1, PD-L2, PD-1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics or small molecules, that bind to and block or inhibit the activity of one or more of CTLA-4, PD-L1, PD-L2, PD-1, BTLA, HVEM, TIM3, GAL9, LAG3, VISTA, KIR, 2B4, CD 160 and CGEN-15049.

Cytotoxic T-lymphocyte-associated protein 4 (CTLA-4) is an immune checkpoint molecule that down-regulates pathways of T-cell activation. CTLA-4 is a negative regulator of T-cell activation. Blockade of CTLA-4 has been shown to augment T-cell activation and proliferation. The combination of the herpes simplex virus and the anti-CTLA-4 antibody is intended to enhance T-cell activation through two different mechanisms in order to augment the anti-tumor immune response to tumor antigen released following the lytic replication of the virus in the tumor. Therefore, the combination of the herpes simplex virus and the anti-CTLA-4 antibody may enhance the destruction of the injected and un-injected/distal tumors, improve overall tumor response, and extend overall survival, in particular where the extension of overall survival is compared to that obtained using an anti-CTLA-4 antibody alone.

Programmed cell death protein 1 (PD-1) is a 288 amino acid cell surface protein molecule expressed on T cells and pro-B cells and plays a role in their fate/differentiation. PD-1's two ligands, PD-L1 and PD-L2, are members of the B7 family. PD-1 limits the activity of T cells in peripheral tissues at the time of an inflammatory response to infection and to limit autoimmunity PD-1 blockade in vitro enhances T-cell proliferation and cytokine production in response to a challenge by specific antigen targets or by allogeneic cells in mixed lymphocyte reactions. A strong correlation between PD-1 expression and response was shown with blockade of PD-1 (Pardoll, Nature Reviews Cancer, 12: 252-264, 2012). PD-1 blockade can be accomplished by a variety of mechanisms including antibodies that bind PD-1 or PD-L1.

Programmed death-ligand 1 (PD-L1) also referred to as cluster of differentiation 274 (CD274) or B7 homolog 1 (B7-H1) is a protein encoded by the CD274 gene. See, Entrez Gene: CD274 CD274 molecule. PD-L1, a 40 kDa type 1 transmembrane protein that plays a role in suppressing the immune system, binds to its receptor (PD-1) found on activated T cells, B cells, and myeloid cells, to modulate cell activation or inhibition. See, Chemnitz et al., Journal of Immunology, 173 (2):945-54 (2004).

Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Also included are B7 inhibitors, such as B7-H3 and B7-H4 inhibitors (e.g., the anti-B7-H3 antibody MGA271 (Loo et al., 2012, Clin. Cancer Res. July 15 (18) 3834). Another checkpoint inhibitor is TIM3 (T-cell immunoglobulin domain and mucin domain 3) (Fourcade et al., 2010, J. Exp. Med. 207:2175-86 and Sakuishi et al., 2010, J. Exp. Med. 207:2187-94).

As described further herein, in one aspect, the present invention relates to the use of combinations of oncolytic viruses and checkpoint inhibitors for the treatment of cancers. In another aspect, the present invention relates to pharmaceutical compositions comprising the combination of the oncolytic viruses and checkpoint inhibitors.

Thus, in one aspect of the present invention, the checkpoint inhibitor is a blocker or inhibitor of CTLA-4, PD-1, PD-L1, or PD-L2. In some embodiments, the checkpoint inhibitor is a blocker or inhibitor of CTLA-4 such as tremelimumab, ipilimumab (also known as 10D1, MDX-D010), BMS-986249, AGEN-1884, and anti-CTLA-4 antibodies described in U.S. Pat. Nos. 5,811,097; 5,811,097; 5,855,887; 6,051,227; 6,207,157; 6,682,736; 6,984,720; and 7,605,238, each of which is incorporated herein by reference. In some embodiments, the checkpoint inhibitor is a blocker or inhibitor of PD-L1 or PD-1 (e.g., a molecule that inhibits PD-1 interaction with PD-L1 and/or PD-L2 inhibitors) such as include pembrolizumab (anti-PD-1 antibody), nivolumab (anti-PD-1 antibody), CT-011 (anti-PD-1 antibody), CX-072 (anti-PD-L1 antibody), IO-103 (anti-PD-L1), BGB-A333 (anti-PD-L1), WBP-3155 (anti-PD-L1), MDX-1105 (anti-PD-L1), LY-3300054 (anti-PD-L1), KN-035 (anti-PD-L1), FAZ-053 (anti-PD-L1), CK-301 (anti-PD-L1), AK-106 (anti-PD-L1), M-7824 (anti-PD-L1), CA-170 (anti-PD-L1), CS-1001 (anti-PD-L1 antibody); SHR-1316 (anti-PD-L1 antibody); BMS 936558 (anti-PD-1 antibody), BMS-936559 (anti-PD-1 antibody), atezolizumab (anti-PD-L1 antibody), AMP 224 (a fusion protein of the extracellular domain of PD-L2 and an IgG1 antibody designed to block PD-L2/PD-1 interaction), MED14736 (durvalumab; anti PD-L1 antibody), MSB0010718C (anti-PD-L1 antibody), and those described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699, each of which is incorporated herein by reference. Additional anti-PD-1 antibodies include PDR-001; SHR-1210; BGB-A317; BCD-100; JNJ-63723283; PF-06801591; BI-754091; JS-001; AGEN-2034; MGD-013; LZM-009; GLS-010; MGA-012; AK-103; genolimzumab; dostarlimab; cemiplimab; IBI-308; camrelizumab; AMP-514; TSR-042; Sym-021; HX-008; and ABBV-368.

BMS 936558 is a fully human IgG4 monoclonal antibody targeting PD-1. In a phase I trial, biweekly administration of BMS-936558 in subjects with advanced, treatment-refractory malignancies showed durable partial or complete regressions. The most significant response rate was observed in subjects with melanoma (28%) and renal cell carcinoma (27%), but substantial clinical activity was also observed in subjects with non-small cell lung cancer (NSCLC), and some responses persisted for more than a year.

BMS 936559 is a fully human IgG4 monoclonal antibody that targets the PD-1 ligand PD-L1. Phase I results showed that biweekly administration of this drug led to durable responses, especially in subjects with melanoma. Objective response rates ranged from 6% to 17%) depending on the cancer type in subjects with advanced-stage NSCLC, melanoma, RCC, or ovarian cancer, with some subjects experiencing responses lasting a year or longer.

AMP 224 is a fusion protein of the extracellular domain of the second PD-1 ligand, PD-L2, and IgG1, which has the potential to block the PD-L2/PD-1 interaction. AMP-224 is currently undergoing phase I testing as monotherapy in subjects with advanced cancer.

MEDI4736 is an anti-PD-L1 antibody that has demonstrated an acceptable safety profile and durable clinical activity in this dose-escalation study. Expansion in multiple cancers and development of MEDI4736 as monotherapy and in combination is ongoing.

### Methods of Treating a Disease or Disorder

The present invention also relates to methods of treating diseases or disorders, such as cancer, with an oncolytic virus (e.g., HSV1/ICP34.5⁻/ICP47⁻/FLT3L/IL12). The oncolytic viruses of the present invention (e.g., HSV1/ICP34.5⁻/ICP47⁻/FLT3L/IL12), can be used to treat any injectable cancer (i.e., any tumor that can be injected with e.g., a needle, with or without guidance (e.g., visual or ultrasound guidance)). In some embodiments, the cancer is B-cell lymphoma (e.g., diffuse large B-cell lymphoma), non-small cell lung cancer, small cell lung cancer, basal cell carcinoma, cutaneous squamous cell carcinoma, colorectal cancer, melanoma (e.g., uveal melanoma), head and neck squamous cancer, hepatocellular cancer, gastric cancer, sarcoma (e.g., soft tissue sarcoma, ewing sarcoma, osteosarcoma, or rhabdomyosarcoma), gastroesophageal cancer, renal cell carcinoma, glioblastoma, pancreatic cancer, bladder cancer, prostate cancer, breast cancer (e.g., triple negative breast carcinoma), cutaneous T-cell lymphoma, merkel cell carcinoma, or multiple myeloma.

The term “metastatic cancer” refers to a cancer that has spread from the part of the body where it started (i.e., the primary site) to other parts of the body. When cancer has spread to a new area (i.e., metastasized), it's still named after the part of the body where it started. For instance, colon cancer that has spread to the pancreas is referred to as “metastatic colon cancer to the pancreas,” as opposed to pancreatic cancer. Treatment is also based on where the cancer originated. If colon cancer spreads to the bones, it's still a colon cancer, and the relevant physician will recommend treatments that have been shown to combat metastatic colon cancer.

The present invention also relates to the use of combinations of oncolytic viruses (e.g., HSV1/ICP34.5⁻/ICP47⁻/FLT3L/IL12) and other agents (e.g., checkpoint inhibitors) for the treatment of cancers such as those discussed above.

The present invention also relates to a method of treating diseases or disorders, such as cancer by administering: (i) a therapeutically effective amount of an oncolytic virus (e.g., HSV1/ICP34.5⁻/ICP47⁻/FLT3L/IL12); and (ii) a therapeutically effective amount of another agent (e.g., a checkpoint inhibitor).

In particular embodiments, the present invention relates to a combination of an oncolytic virus (e.g., HSV1/ICP34.5⁻/ICP47⁻/FLT3L/IL12) and an anti-PD-1 antibody, an oncolytic virus (e.g., HSV1/ICP34.5⁻/ICP47⁻/FLT3L/IL12) and an anti-PD-L1 antibody, or an oncolytic virus (e.g., HSV1/ICP34.5⁻/ICP47⁻/FLT3L/IL12) and an anti-CTLA-4 antibody. In specific embodiments, the oncolytic virus is HSV1/ICP34.5⁻/ICP47⁻/FLT3L/IL12.

In many instances, cancer is present in patients as both a primary tumor (i.e., a tumor growing at the anatomical site where tumor progression began and proceeded to yield a cancerous mass) and as a secondary tumor or metastasis (i.e., the spread of a tumor from its primary site to other parts of the body). The oncolytic viruses of the present invention can be efficacious in treating tumors via a lytic effect and systemic immune effect. For example, HSV1/ICP34.5⁻/ICP47⁻/FLT3L/IL12 physically lyses tumors cells causing primary tumor cell death and the release of tumor-derived antigens which are then recognized by the immune system. In addition, replication of HSV1/ICP34.5⁻/ICP47⁻/FLT3L/IL12 results in the production of FLT3L and IL12 which aids in the mounting and maintenance of anti-tumor immune response (both locally and systemically) such that the immune system can recognize and attack both the primary and secondary tumors/metastases. Accordingly, the present invention contemplates the treatment of primary tumors, metastases (i.e., secondary tumors), or both with an oncolytic virus (e.g., HSV1/ICP34.5⁻/ICP47⁻/FLT3L/IL12) either alone or in combination with a second agent (e.g., a checkpoint inhibitor).

In some embodiments, the methods of treatment or uses described herein include a combination treatment with targeted cancer therapies, e. g., MEK inhibitors such as cobimetinib, trametinib, and binimetinib. In other embodiments, the methods of treatment or uses described herein include treatment with cytokines, such as pegylated IL2 (e.g., bempegaldesleukin) or pegylated IL10 (e.g., pegilodecakin). In yet other embodiments, the methods of treatment or uses described herein include treatment with a combination of targeted therapy and immune modulators.

The methods of the present invention can be used to treat several different stages of cancer. Most staging systems include information relating to whether the cancer has spread to nearby lymph nodes, where the tumor is located in the body, the cell type (e.g., squamous cell carcinoma), whether the cancer has spread to a different part of the body, the size of the tumor, and the grade of tumor (i.e., the level of cell abnormality the likelihood of the tumor to grow and spread). For example, Stage 0 refers to the presence of abnormal cells that have not spread to nearby tissue—i.e., cells that may become a cancer. Stage I, Stage II, and Stage III cancer refer to the presence of cancer. The higher the Stage, the larger the cancer tumor and the more it has spread into nearby tissues. Stage IV cancer is cancer that has spread to distant parts of the body. In some embodiments, the methods of the present invention can be used to treat metastatic cancer.

### Pharmaceutical Compositions

The present invention also relates to pharmaceutical compositions comprising oncolytic viruses (e.g., HSV1/ICP34.5⁻/ICP47⁻/FLT3L/IL12), or comprising the combination of the oncolytic viruses (e.g., HSV1/ICP34.5⁻/ICP47⁻/FLT3L/IL12) and checkpoint inhibitors, targeted cancer therapies, and/or other immune modulators. The pharmaceutical composition may contain formulation materials for modifying, maintaining or preserving, for example, the pH, osmolarity, viscosity, clarity, color, isotonicity, odor, sterility, stability, rate of dissolution or release, adsorption, or penetration of the composition. Pharmaceutically active agents can be administered to a patient by various routes including, for example, orally or parenterally, such as intravenously, intramuscularly, subcutaneously, intraorbitally, intracapsularly, intraperitoneally, intrarectally, intracisternally, intratumorally, intravasally, intradermally or by passive or facilitated absorption through the skin using, for example, a skin patch or transdermal iontophoresis, respectively. In one embodiment, the oncolytic virus (e.g., HSV1/ICP34.5⁻/ICP47⁻/FLT3L/IL12) is injected into the tumor (i.e., via intratumoral injection). In another embodiment, the checkpoint inhibitor (e.g., an anti-PD-1 antibody, anti-PD-L1 antibody, or anti-CTLA-4 antibody) is administered systemically (e.g., intravenously). In another embodiment, the targeted therapy (e.g., MEK small molecule kinase inhibitor, such as cobimetinib, trametinib, or binimetinib) is administered systemically via oral route. In yet another embodiment, the cytokines, such as pegylated IL2 (e.g., bempegaldesleukin) or pegylated IL10 (e.g., pegilodecakin), is administered systemically.

One of ordinary skill in the art would be able to determine the dosage and duration of treatment according to any aspect of the present disclosure. For example, the skilled artisan may monitor patients to determine whether treatment should be started, continued, discontinued or resumed. An effective amount for a particular patient may vary depending on factors such as the condition being treated, the overall health of the patient and the method, route and dose of administration. The clinician using parameters known in the art makes determination of the appropriate dose. An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding agent molecule is being used, the route of administration, and the size (body weight, body surface or organ size) and condition (the age and general health) of the patient. Accordingly, the clinician may titer the dosage and modify the route of administration to obtain the optimal therapeutic effect.

Clinical studies have demonstrated that oncolytic viruses can be injected directly into cutaneous, subcutaneous or nodal lesions that are visible, palpable, or can be injected with ultrasound-guidance. Thus, in one aspect, pharmaceutical compositions comprising HSV1/ICP34.5⁻/ICP47⁻/FLT3L/IL12 are administered via intralesional injection. In some embodiments, HSV1/ICP34.5⁻/ICP47⁻/FLT3L/IL12 is provided in 1 mL single-use vials in fixed dosing concentrations: 10⁶ pfu/mL for initial dosing and 10⁸ pfu/mL for subsequent dosing. The volume that is injected may vary depending on the tumor type. For example, HSV1/ICP34.5⁻/ICP47⁻/FLT3L/IL12 may be administered by intratumoral injection into injectable cutaneous, subcutaneous, and nodal tumors at a dose of up to 4.0 mL of 10⁶ plaque forming unit/mL (PFU/mL) at day 1 of week 1 followed by a dose of up to 4.0 mL of 10⁸ PFU/mL at day 1 of week 4, and every 2 weeks (±3 days) thereafter. In another embodiment, HSV1/ICP34.5⁻/ICP47⁻/FLT3L/IL12 is administered by intratumoral injection into injectable cutaneous, subcutaneous, and nodal tumors at a dose of up to 4.0 mL of 10⁶ plaque forming unit/mL (PFU/mL) at day 1 of week 1 followed by a dose of up to 4.0 mL of 10⁷ PFU/mL at day 1 of week 4, and every 2 weeks (±3 days) thereafter.

Compositions of the present invention may comprise one or more additional components including a physiologically acceptable carrier, excipient or diluent. For example, the compositions may comprise one or more of a buffer, an antioxidant such as ascorbic acid, a low molecular weight polypeptide (e.g., having fewer than 10 amino acids), a protein, an amino acid, a carbohydrate such as glucose, sucrose or dextrins, a chelating agent such as EDTA, glutathione, a stabilizer, and an excipient. Acceptable diluents include, for example, neutral buffered saline or saline mixed with specific serum albumin. Preservatives such as benzyl alcohol may also be added. The composition may be formulated as a lyophilizate using appropriate excipient solutions (e.g., sucrose) as diluents.

In certain embodiments, the checkpoint inhibitor is administered in 0.01 mg/kg, 0.05 mg/kg, 0.1 mg/kg, 0.2 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, or any combination thereof doses. In certain embodiments the checkpoint inhibitor is administered once a week, twice a week, three times a week, once every two weeks, or once every month. In certain embodiments, the checkpoint inhibitor is administered as a single dose, in two doses, in three doses, in four doses, in five doses, or in 6 or more doses.

In certain embodiments, the anti-PD-1 antibody is administered by injection (e.g., subcutaneously or intravenously) at a dose of about 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, or 4 weeks. In one embodiment, the anti-PD-1 antibody is administered at a dose from about 10 to 20 mg/kg every other week.

In one embodiment, the anti-PD-1 antibody molecule, e.g., nivolumab, is administered intravenously at a dose from about 1 mg/kg to 3 mg/kg, e.g., about 1 mg/kg, 2 mg/kg or 3 mg/kg, every two weeks. In one embodiment, the anti-PD-1 antibody molecule, e.g., nivolumab, is administered intravenously at a dose of about 2 mg/kg at 3-week intervals. In one embodiment, nivolumab is administered in an amount from about 1 mg/kg to 5 mg/kg, e.g., 3 mg/kg, and may be administered over a period of 60 minutes, ca. once a week to once every 2, 3 or 4 weeks.

In one embodiment, the anti-PD-1 antibody molecule, e.g., pembrolizumab, is administered intravenously at a dose from about 1 mg/kg to 3 mg/kg, e.g., about 1 mg/kg, 2 mg/kg or 3 mg/kg, every three weeks. In one embodiment, the anti-PD-1 antibody molecule, e.g., pembrolizumab, is administered intravenously at a dose of about 2 mg/kg at 3-week intervals. In another embodiment, the anti-PD-1 antibody molecule, e.g., pembrolizumab, is administered intravenously at a dose from about 100 mg/kg to 300 mg/kg, e.g., about 100 mg/kg, 200 mg/kg or 300 mg/kg, every three weeks. In one embodiment, the anti-PD-1 antibody molecule, e.g., pembrolizumab, is administered intravenously at a dose of about 200 mg/kg at 3-week intervals.

In certain embodiments, the anti-CTLA-4 antibody (e.g., ipilimumab) is administered by injection (e.g., subcutaneously or intravenously) at a dose of about 3 mg/kg IV Q3W for a maximum of 4 doses; about 3 mg/kg IV Q6W for a maximum of 4 doses; about 3 mg/kg IV Q12W for a maximum of 4 doses; about 10 mg/kg IV Q3W for a maximum of 4 doses; or about 10 mg/kg IV Q12W for a maximum of 4 doses. In certain embodiments, the anti-CTLA-4 antibody (e.g., tremelimumab) is administered by injection (e.g., subcutaneously or intravenously) at a dose of about 10 mg/kg Q4W; or about 15 mg/kg every 3 months.

In certain embodiments, the anti-PD-L1 antibody (e.g., atezolizumab) is administered by injection (e.g., subcutaneously or intravenously) at a dose of about 1200 mg IV Q3W until disease progression or unacceptable toxicity.

Thus, in one embodiment, the present invention relates to a pharmaceutical composition for use in a method of treating any injectable cancer. In some embodiments, the cancer is B-cell lymphoma (e.g., diffuse large B-cell lymphoma), non-small cell lung cancer, small cell lung cancer, basal cell carcinoma, cutaneous squamous cell carcinoma, colorectal cancer, melanoma (e.g., uveal melanoma), head and neck squamous cancer, hepatocellular cancer, gastric cancer, sarcoma (e.g., soft tissue sarcoma, ewing sarcoma, osteosarcoma, or rhabdomyosarcoma), gastroesophageal cancer, renal cell carcinoma, glioblastoma, pancreatic cancer, bladder cancer, prostate cancer, breast cancer (e.g., triple negative breast carcinoma), cutaneous T-cell lymphoma, merkel cell carcinoma, or multiple myeloma, wherein the pharmaceutical composition comprises an oncolytic virus (e.g., HSV1/ICP34.5⁻/ICP47⁻/FLT3L/IL12), or an oncolytic virus (e.g., HSV1/ICP34.5⁻/ICP47⁻/FLT3L/IL12) and a second agent (e.g., a checkpoint inhibitor).

In other embodiments, the present invention relates to a therapeutically effective amount of an oncolytic virus (e.g., HSV1/ICP34.5⁻/ICP47⁻/FLT3L/IL12) for use in treating B-cell lymphoma (e.g., diffuse large B-cell lymphoma), non-small cell lung cancer, small cell lung cancer, basal cell carcinoma, cutaneous squamous cell carcinoma, colorectal cancer, melanoma (e.g., uveal melanoma), head and neck squamous cancer, hepatocellular cancer, gastric cancer, sarcoma (e.g., soft tissue sarcoma, ewing sarcoma, osteosarcoma, or rhabdomyosarcoma), gastroesophageal cancer, renal cell carcinoma, glioblastoma, pancreatic cancer, bladder cancer, prostate cancer, breast cancer (e.g., triple negative breast carcinoma), cutaneous T-cell lymphoma, merkel cell carcinoma, or multiple myeloma. In yet other embodiments, the present invention relates to a therapeutically effective amount of an oncolytic virus (e.g., HSV1/ICP34.5⁻/ICP47⁻/FLT3L/IL12) and a second agent (e.g., a checkpoint inhibitor) for use in treating B-cell lymphoma (e.g., diffuse large B-cell lymphoma), non-small cell lung cancer, small cell lung cancer, basal cell carcinoma, cutaneous squamous cell carcinoma, colorectal cancer, melanoma (e.g., uveal melanoma), head and neck squamous cancer, hepatocellular cancer, gastric cancer, sarcoma (e.g., soft tissue sarcoma, ewing sarcoma, osteosarcoma, or rhabdomyosarcoma), gastroesophageal cancer, renal cell carcinoma, glioblastoma, pancreatic cancer, bladder cancer, prostate cancer, breast cancer (e.g., triple negative breast carcinoma), cutaneous T-cell lymphoma, merkel cell carcinoma, or multiple myeloma.

### Kits

In another aspect, the present invention relates to kits comprising [1] the oncolytic virus (e.g., HSV1/ICP34.5⁻/ICP47⁻/FLT3L/IL12), optionally in combination with a second agent (e.g., a checkpoint inhibitor); and [2] instructions for administration to patients. For example, a kit of the present invention may comprise an oncolytic virus (e.g., HSV1/ICP34.5⁻/ICP47⁻/FLT3L/IL12), and instructions (e.g., in a package insert or label) for treating a patient with cancer. In some embodiments, the cancer is a metastatic cancer. In another embodiment, the kit of the present invention may comprise an oncolytic virus (e.g., HSV1/ICP34.5⁻/ICP47⁻/FLT3L/IL12), a checkpoint inhibitor (e.g., an anti-PD-1 antibody, anti-PD-L1 antibody, or anti-CTLA-4 antibody), and instructions (e.g., in a package insert or label) for treating a patient with cancer.

In some embodiments, the second agent is a targeted cancer therapy (e g., MEK inhibitor such as cobimetinib, trametinib, and binimetinib) or a cytokine (e.g., pegylated IL2 (e.g., bempegaldesleukin) or pegylated IL10 (e.g., pegilodecakin)).

In some embodiments, the kit comprising HSV1/ICP34.5⁻/ICP47⁻/FLT3L/IL12 comprises instructions (e.g., in a package insert or label) for administration by intratumoral injection at a dose of up to 4.0 ml of 10⁶ PFU/mL at day 1 of week 1 followed by a dose of up to 4.0 ml of 10⁸ PFU/mL at day 1 of week 4, and every 2 weeks thereafter (e.g., until complete response). In some embodiments, the kit comprising HSV1/ICP34.5⁻/ICP47⁻/FLT3L/IL12 comprises instructions (e.g., in a package insert or label) for administration by intratumoral injection at a dose of up to 4.0 ml of 10⁶ PFU/mL at day 1 of week 1 followed by a dose of up to 4.0 ml of 10⁷ PFU/mL at day 1 of week 4, and every 2 weeks thereafter (e.g., until complete response).

In embodiments where the kit comprises an anti-PD-1 antibody, the kit comprises instructions (e.g., in a package insert or label) for intravenous administration at doses described herein. Examples of anti-PD-1 antibodies include, pembrolizumab and nivolumab.

In embodiments where the kit comprises an anti-PD-L1 antibody, the kit comprises instructions (e.g., in a package insert or label) for intravenous administration at doses described herein. Examples of anti-PD-L1 antibodies include, atezolizumab.

In embodiments where the kit comprises an anti-CTLA-4 antibody, the kit comprises instructions (e.g., in a package insert or label) for intravenous administration at doses described herein. Examples of anti-CTLA-4 antibodies include, ipilimumab.

In another embodiment is provided a method of manufacturing the kits of the present invention.

### EXAMPLES

The following examples are provided for the purpose of illustrating specific embodiments or features of the present invention and are not intended to limit its scope.

### Example 1: Interleukin-12 (IL12) Produced as a Single Chain Protein with the p40 Subunit in the 5′ Position and the p35 Subunit in the 3′ Position and Connected Via a Single G4S Linker is Active In Vitro and In Vivo

An engineered single chain IL12 molecule with specific engineering criteria results in optimal expression and activity of the cytokine.

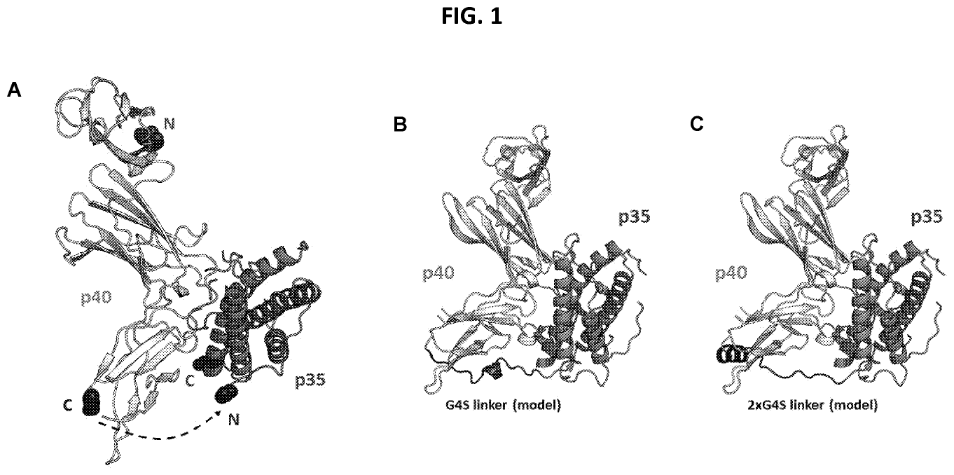

The optimal configuration of the p40 and p35 subunits of IL12 was evaluated by analyzing the crystal structure of IL12 (PDB ID 3HMX). A single chain protein is expected to have a higher degree of heterodimerization efficiency as the subunits are in proximity for assembly. The p40-p35 orientation (FIG. 1A; dashed lines) is structurally preferred over the p35-p40 orientation due to proximity of C- and N-termini connection points. This results in a linker that spans a ~36 angstrom gap (connecting the carboxy terminal end of p40 to the amino initiation end of p35). In contrast, the generation of a p35-p40 peptide results in a ~60 angstrom gap which requires a longer linker and is less favorable.

To model linkers between the p40 and p35 subunits, the p40 and p35 subunits of the crystal structure of IL12 (PDB 3HMX) was prepared using FastRelax with 0.5 Å coordinate constraints in RosettaScripts (S. J. Fleishman, A. Leaver-Fay, J. E. Corn, E.-M. Strauch, S. D. Khare, N. Koga, J. Ashworth, P. Murphy, F. Richter, G. Lemmon, J. Meiler and D. Baker. RosettaScripts: A Scripting Language Interface to the Rosetta Macromolecular Modeling Suite. PLoS ONE. 2011, 6, 6, e20161). The resulting PDB file was concatenated into a single chain with the orientation p40-p35 and then Rosetta Remodel was used to model the following linkers between the two domains: an elastin-based linker that has been described previously (VPGVGVPGVGGS), G4S (FIG. 1B), 2×(G4S) (FIG. 1C), 3×(G4S), and no linker. The unresolved the C-terminal residue of p40 (S340) and first 11 residues of mature p35 (RNLPVATPDPG) were included in the Remodel runs. A control lacking the unresolved residues was also run. Linkers were expected to be required as the calculated rate of loop closure using Rosetta loop modeling simulations was significantly improved when linkers were incorporated. For each linker, 2880 Remodel trajectories were run using fragment insertion from loop fragments for sampling and CCD-based inverse kinematics for loop closure. Models were scored with the Remodel weights set and models with successful loop closures (chain break score<0.07) were output as PDB files. Loop closure rates were determined by evaluating the percentage of trajectories meeting the loop closure criteria. For each linker, conformational convergence was measured by plotting the RMSD of each model to the lowest scoring model using the RMSD Mover in RosettaScripts without superposition. The top ten models for each linker were evaluated by Rosetta Energy Units (REU) per residue and by backbone score terms for linker residues (Table 1). Models with Ramachandran outliers were identified in MOE (Chemical Computing Group, Inc.).

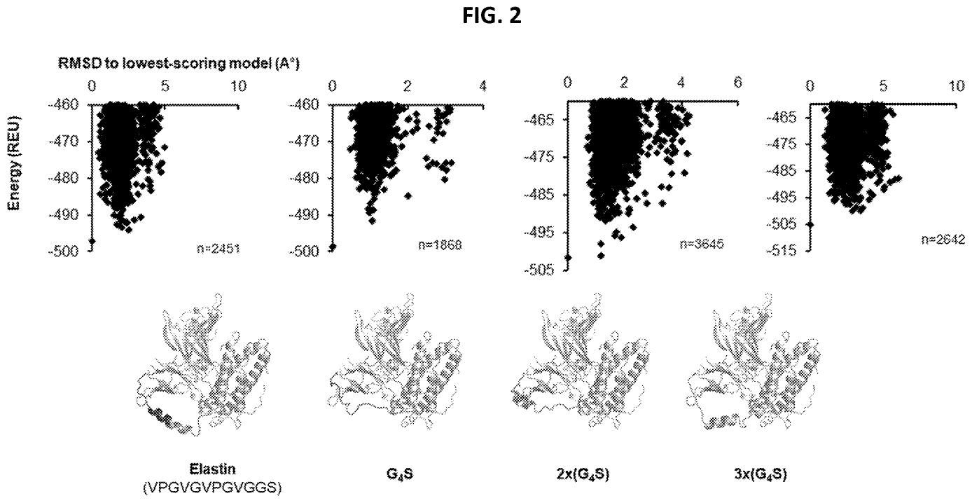

The Remodel runs with no linker or with truncated unresolved p40 and p35 termini had loop closure rates<10%, suggesting that a linker is necessary to link the p40 and p35 subunits as a single chain. In contrast, Remodel runs with linkers had successful loop closure rates for all four linker sequences. Top scoring models for all four linkers scored well without backbone strain or Ramachandran outliers. The longer elastin and 3×(G₄S) linkers are likely to be more conformationally flexible than the G₄S and 2×(G₄S) linkers, as models from the former showed a greater RMSD divergence from the top-scoring model than models from the latter. Rosetta Remodel was used to identify linkers for the p40-linker-p35 payload. Top scoring models of the G4S-linked and 2×G4S-linked constructs suggest that both linkers were suitable, as was the elastin-based linker (FIG. 2).

The loop closure rates are summarized in Table 1, below.

TABLE 1
Rate of loop closure for linkers evaluated
for fusion of IL12p35 and IL12p40 chains.

| | | | | | | No |
|---|---|---|---|---|---|---|
| | | | | | No | disordered |
| | elastin | G₄S | 2x(G₄S) | 3x(G₄S) | linker | regions |
| Run 1 | 28 | 19 | 316 | 312 | 13 | 0 |
| Run 2 | 14 | 151 | 13 | 310 | 35 | 0 |
| Run 3 | 13 | 187 | 175 | 318 | 41 | 0 |
| Run 4 | 317 | 25 | 23 | 19 | 50 | 0 |
| Run 5 | 317 | 138 | 319 | 14 | 9 | 0 |
| Run 6 | 27 | 166 | 314 | 18 | 32 | 0 |
| Run 7 | 243 | 313 | 178 | 317 | 23 | 0 |
| Run 8 | 318 | 10 | 315 | 315 | 64 | 0 |
| Run 9 | 14 | 310 | 299 | 19 | 7 | 0 |
| Total | 1291 | 1319 | 1952 | 1642 | 274 | 0 |
| Percent | 44.8 | 45.8 | 67.8 | 57.0 | 9.5 | 0.0 |
| loop closure | | | | | | |
| success (%) | | | | | | |

To confirm the function of the single chain IL12 from the in silico modeling, the single-chain IL12 constructs in various formats were cloned into pΔ34.5(XS) vector (see construct depiction, FIG. 3A), a pcDNA3.1 based vector with the construct inserted between a CMV promoter and a BGH poly(A) tail. The HSV-1 inverted repeats flanking CMV promoter and BGH poly(A) tail facilitates the recombination of the single chain IL12 constructs, CMV and BGH poly(A) tail into the HSV-1 virus. pΔ34.5(XS) vector was linearized by restriction enzymes Hind III and Xho I, which are located after the CMV promoter and preceding BGH poly(A) tail respectively. Overlapping DNA fragments encoding the single-chain IL12 constructs were ordered and cloned into the linearized pΔ34.5(XS) vector using Gibson assembly method. The authenticity of the single-chain IL12 constructs was confirmed by DNA sequencing. These constructs were used to transfect HEK 293 cells in vitro and compare IL12 protein production. Cells were transfected with 4 μg DNA with 8 μl of lipofectamine 2000 in Optimem media and incubated for 48 hours at 37° C. with 5% CO₂. Supernatants were removed and IL12 expression was quantitated using a Biolegend human IL12p70 ELISA assay. The position of the peptide chains significantly altered expression. The construct containing p35-elastin-p40 did not produced detectable levels of IL12 whereas the construct containing p40-elastin-p35 produced IL12 (FIG. 3B).

In native form, IL12 is produced as two independent chains, both of which contain signal peptides required for protein secretion. In the modified version, the necessity of the second signal peptide was evaluated. A construct containing a single signal peptide located at the 5′ end of the fusion [IL12(p40-elastin-No SP-p35)] was compared with a construct encoding signal peptides in both the p35 and p40 subunits [IL12(p40-elastin-p35)]. The removal of the second signal peptide increased the overall yield of IL12 produced as a result of the transfection (FIG. 3B). Finally, the expression of IL12 with an elastin linker was compared to a single G4S linker (FIG. 3B). Based on these observations, a single chain IL12 cassette incorporating a p40-G4S linker-p35 with the signal peptide removed from the p35 subunit was selected for inclusion into the engineered virus.

### Example 2: Bioactive FLT3L and IL12 are Expressed Simultaneously Via the Addition of a P2A Linker

These experiments relate to the engineering performed to produce bioactive FLT3L and IL12 in a bicistronic format under the control of a single promoter using a porcine tescho 2A sequence.

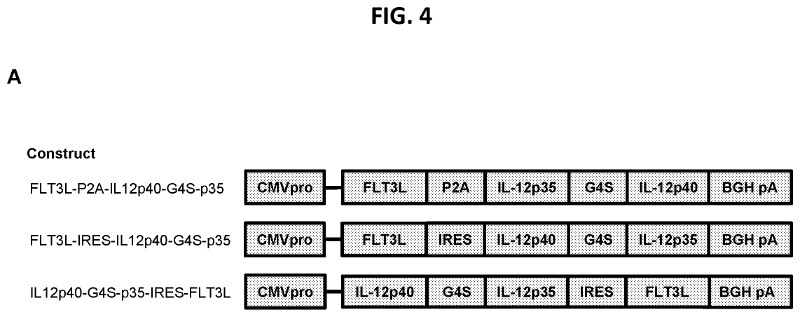

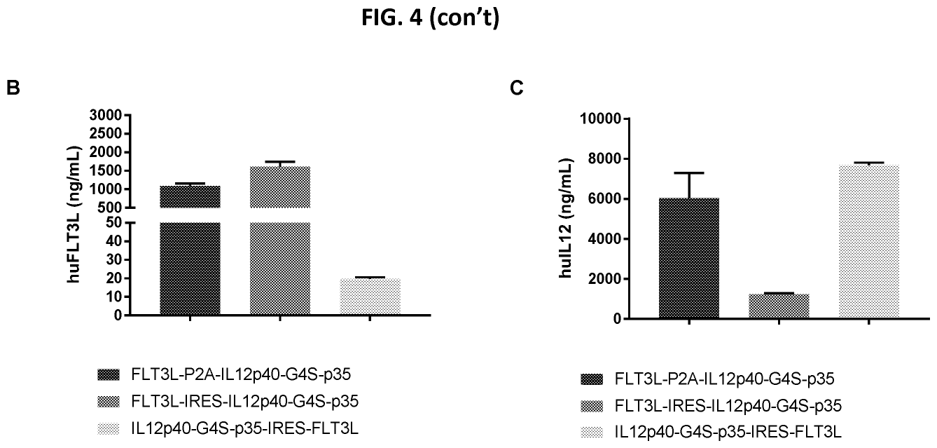

The expression of multiple, rationally selected, proteins from a virus should enhance the immunostimulatory capacity of the virus to elicit an anti-tumor response. FLT3L and IL12 were selected as immunostimulatory cytokines. A single promoter (CMV promoter) was used to produce both cytokines. This approach had the benefit of producing both cytokines in the same infected cell at the same rate and at the same time. We selected two means to express multiple proteins from a single promoter: internal ribosomal entry sites (IRES) and 2A sequences. DNA constructs were designed incorporating FLT3L-IRES-IL12, IL12-IRES-FLT3L or FLT3L-P2A-IL12. The DNA constructs were tested in vitro as previously described (FIG. 4A). DNA constructs were transfected in 293T cells and supernatants were tested by ELISA (Biolegend IL12p70 assay for IL12 and Thermo FLT3L assay for FLT3L).

In either orientation (FLT3L as the first gene and IL12 as the second, or (IL12 as the first gene and FLT3L as the second), the production of the second gene was decreased when using the IRES (FIGS. 4B and 4C). For this reason, the P2A sequence was chosen as the functional unit to provide production of two proteins from a single promoter.

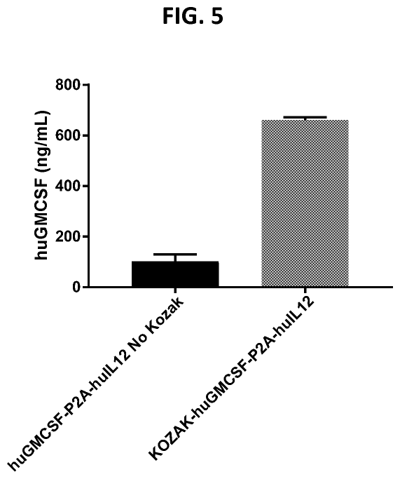

In separate experiments using an alternate payload (GMCSF), the effect of a consensus KOZAK sequence was evaluated. KOZAK sequences are known to enhance mammalian translation and were expected to improve translation of the full cassette. Consistent with this, the expression of the 5′ protein (GMCSF) was significantly increased by the incorporation of a KOZAK sequence upstream of the translational start site independent of the P2A or IRES usage (FIG. 5; (avg ng/ML with KOZAK=660.9; avg ng/mL without KOZAK=102.5)).

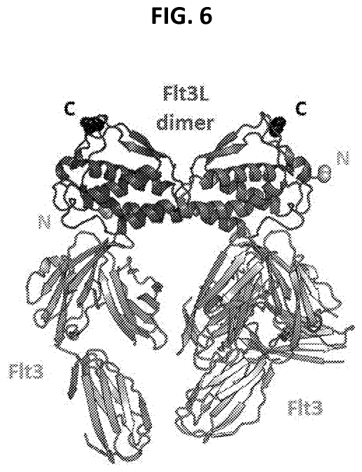

A potential consequence of the addition of the P2A site is that it appends several amino acids to the end of the FLT3L protein. P2A is a sequence that results in the production of two distinct polypeptide chains in the majority of mammalian cells but the first peptide generated includes the addition of the amino acid sequence GSGATNFSLLKQAGDVEENPG. In silico modeling was performed to determine if the addition of amino acids to the carboxy terminal end of FLT3L would affect interaction with its receptor, FLT3. PyMOL v. 1.8.6.0 was used to evaluate the structure of the Flt3L/Flt3 complex to choose the construct orientation in the dual payload vector payload1-P2A-payload2 cassette. P2A results in an 18 amino acid peptide fused to the C-terminus of payload1. The structure of Flt3L/Flt3 reveals the C-terminus of Flt3L to be exposed and distal to the receptor binding site and Flt3L dimerization interface. Flt3L is therefore likely to tolerate the P2A tag and was selected as the payload upstream of the P2A sequence (FIG. 6). However, demonstrating the bio-activity of both FLT3L and IL12 was performed to verify activity.

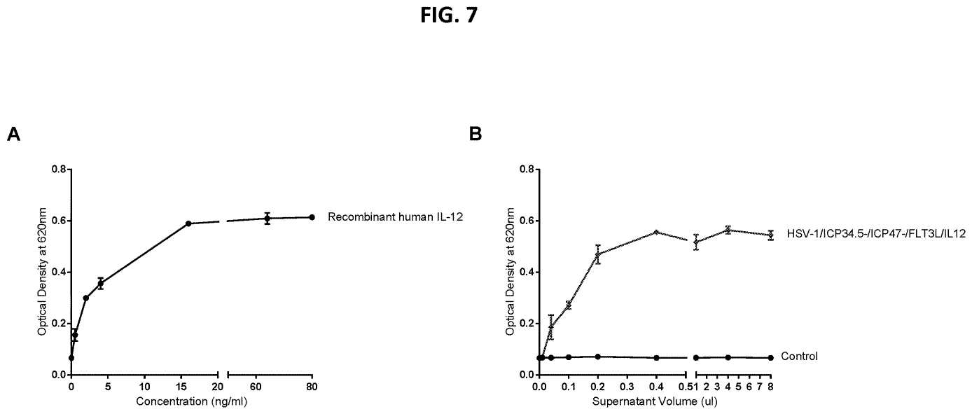

For IL12, the supernatants described previously and used in ELISA assays to quantitate total IL12 expressed were used in an IL12 cell reporter assay. The bioactivity of IL12 was measured using HEK-Blue IL12 cells (Invivogen #hkb-il12). Bio-active IL12 induces the dose-dependent production of secreted embryonic alkaline phosphatase (SEAP) by the HEK-Blue IL12 cell line, and the levels of SEAP can be assessed using a chromogenic reagent, QUANTI-Blue (Invivogen #rep-qb1). Supernatant from DNA-transfected 293T cells were added directly to a 96 well flat bottom plate in three-fold serial dilution in duplicate with HEK-Blue IL12 cells and incubated overnight at 37° C. in 5% CO₂. The following day, QUANTI-Blue reagent was prepared fresh according to manufacturer's instructions, pre-warmed to 37° C. for 15 min, and incubated with 20 μL of overnight cell culture supernatant for 1 h at 37° C. SEAP levels were detected by measuring absorbance at 620-630 nm using a BioTek Synergy Neo2 Microplate Reader (BioTek; Gen5 software v3.04). The supernatants demonstrated activity in the IL12 reporter assay comparable to recombinant human IL12 protein purchased from a commercial vendor (R&D #219-IL-005; FIG. 7).

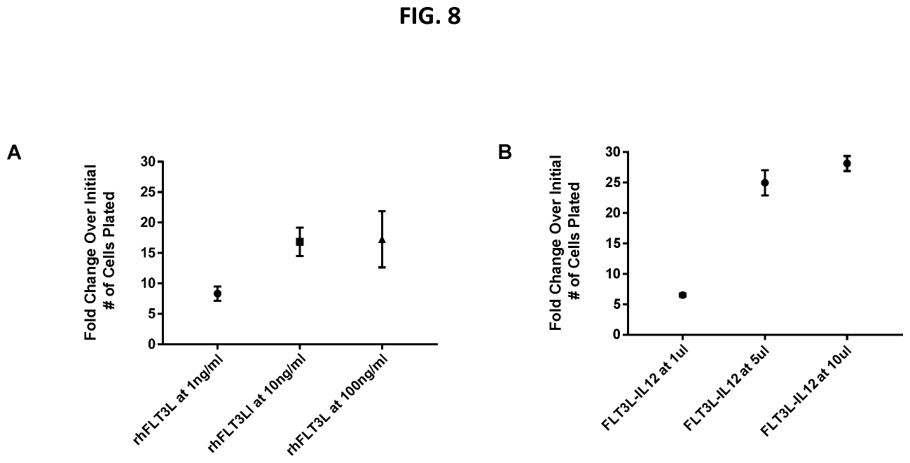

For FLT3L, the supernatants were also tested in a BaF3 cell proliferation assay which has been described in the literature to be a FLT3L sensitive cell line. BaF3 cells were plated at 30,000 cells per well in a 24 well plate in RPMI+10% FBS+geneticin overnight at 37° C. Supernatant from cells transfected with DNA constructs containing the engineered payloads or recombinant human FLT3L was added to the cells, and the total volume was adjusted to 500 uL for all wells before incubating for 14 days at 37° C. in 5% CO₂. On day 14, BaF3 cells were gently resuspended by pipetting, and a sample removed from each well for cell counting using the Vi-CELL XR Cell Viability Analyzer (Beckman Coulter). The total number of viable cells in the well was calculated from the viable cell concentration provided by the Vi-CELL XR. Human recombinant FLT3L was included as a control and the supernatant from transfected 293T cells showed comparable effects on cellular proliferation (FIG. 8).

Based on these observations, the final construct to be recombined into the HSV1 genome was selected as human FLT3L-P2A-huIL12(p40-G₄S-p35) with the engineering described above.

### Example 3: Generation of HSV1/ICP34.5⁻/ICP47⁻/FLT3L/IL12 Virus

The HSV1/ICP34.5⁻/ICP47⁻/FLT3L/IL12 was generated as follows.

### Description of the Viral Genome:

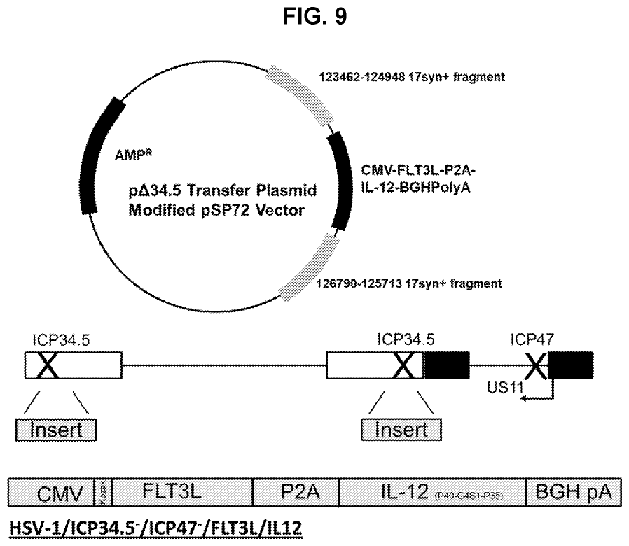

The HSV-1 was derived from strain JS1 as deposited at the European collection of cell cultures (ECAAC) under accession number 01010209. In HSV-1/ICP34.5⁻/ICP47⁻/FLT3L/IL12, the HSV-1 viral genes encoding ICP34.5 and ICP47 have been functionally deleted as described previously. See, Liu et al., Gene Ther., 10:292-303, 2003; U.S. Pat. Nos. 7,223,593 and 7,537,924. In HSV-1/ICP34.5⁻/ICP47⁻/FLT3L/IL12, the functional deletion of the ICP34.5 and ICP47 encoding genes in combination with the early expression of US11 improves tumor replication while maintaining safety. The coding sequences for human FLT3L and IL12 were inserted into the viral genome at the two former sites of the ICP34.5 genes of HSV-1/ICP34.5⁻/ICP47⁻/FLT3L/IL12 (FIG. 9). The human FLT3L and IL12 expression cassette replaces nearly all of the ICP34.5 gene, ensuring that any potential recombination event between HSV-1/ICP34.5⁻/ICP47⁻/FLT3L/IL12 and wild-type virus could only result in a disabled, non-pathogenic virus and could not result in the generation of wild-type virus carrying the genes for human FLT3L and IL12. The HSV thymidine kinase (TK) gene remains intact in HSV-1/ICP34.5⁻/ICP47⁻/FLT3L/IL12, which renders the virus sensitive to anti-viral agents such as acyclovir. Therefore, acyclovir can be used to block HSV-1/ICP34.5⁻/ICP47⁻/FLT3L/IL12 replication, if necessary.

Creation of the pΔ34.5 Transfer Plasmid:

The transfer plasmid containing the human FLT3L and IL12 expression cassette was created from a modified SP72 vector (Promega) as previously described (See, Liu et al., Gene Ther., 10:292-303, 2003; U.S. Pat. Nos. 7,223,593 and 7,537,924). The plasmid contains a modified Sau3AI fragment of HSV-1 17syn+ (nucleotides 123462-126790 with a NotI fragment encoding the majority of ICP34.5 (nucleotides 124948-125713) removed. An expression cassette containing CMV-KOZAK-FLT3L-P2A-IL12-BGHPolyA was inserted into the plasmid near the original NotI site. The insertion results in the expression cassette being flanked by the HSV-1 17syn+ regions excised by the Sau3AI fragment (FIG. 9).

Insertion of Therapeutic Genes into HSV-1/ICP34.5⁻/ICP47⁻/FLT3L/IL12:

Genes were inserted into the viral genome by a process of homologous recombination. Vero cells were transfected with the pΔ34.5 transfer plasmid. The transfected cells were then infected with HSV-1/ICP34.5⁻/ICP47⁻/GFP (JS1 Strain). This virus contained GFP in the ICP34.5 encoding regions of the genome where the CMV-FLT3L-P2A-IL12-BGHPolyA expression cassette was inserted. The transfection-infection reaction was allowed to continue until full CPE (cytopathic effect) was observed. Cells and supernatants from the transfection-infection reaction were diluted and used to infect Vero cells in 96 well plates. After 2 days, the supernatants were evaluated by ELISA to identify wells containing virions expressing IL12 and FLT3L. Cells and supernatants from IL12 and FLT3L positive wells were collected and plated in a plaque assay with Vero cells. After 2 days, recombinant viruses were identified by the loss of the GFP marker gene. The loss of the GFP marker gene suggested GFP at the ICP34.5 sites was replaced by the [CMV]-[Kozak]-[Flt3L]-[P2A]-[IL12]-[BGHpA] expression cassette (FIG. 9). Non-GFP plaques were identified under a fluorescent microscope and they were transferred to an eppendorf tube containing fresh growth medium using a sterile pipette tip. The virus was released from the cells by freeze-thaw and the virus was plated onto new cells. This process was repeated every 2 to 3 days until a homogenous population was achieved (i.e., none of the plaques were green). The insertion of the CMV-FLT3L-P2A-IL12-BGHPolyA expression cassette was validated by PCR and sequencing.

### Example 4: HSV-1/ICP34.5⁻/ICP47⁻/FLT3L/IL12 Virus is Capable of Infecting, Replicating within, and Killing Tumor Cell Lines and Producing Bio-Active FLT3L and IL12 In Vitro

The ability of the recombined virus to maintain cellular infection, replication and lysis while producing bio-active FLT3L and IL12 was evaluated.

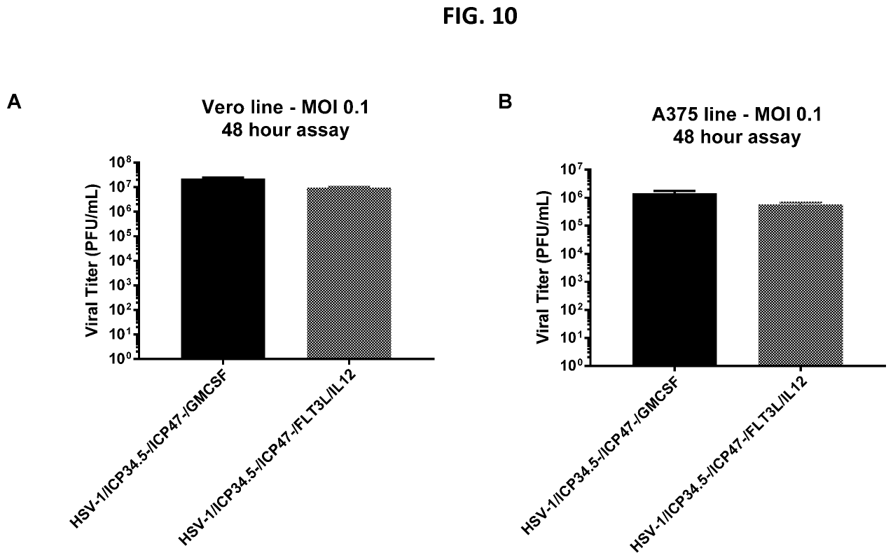

To confirm that the engineered virus was capable of replicating within human cells, two human cell lines were infected and the total amount of virus post infection was quantitated. 1 million A375 or VERO cells were plated in a 6 well dish and incubated overnight at 37° C. in 5% CO₂ in DMEM containing 5% FBS. Cells were infected with HSV-1/ICP34.5⁻/ICP47⁻/FLT3L/IL12 virus at an MOI of 0.1 in triplicate and returned to the incubator. 48 hours post infection, the cells and supernatants were collected and the viral titer was evaluated by plaque assay on Vero cells. The engineered HSV-1/ICP34.5⁻/ICP47⁻/FLT3L/IL12 virus and HSV-1/ICP34.5⁻/ICP47⁻/GMCSF virus were evaluated (FIG. 10).

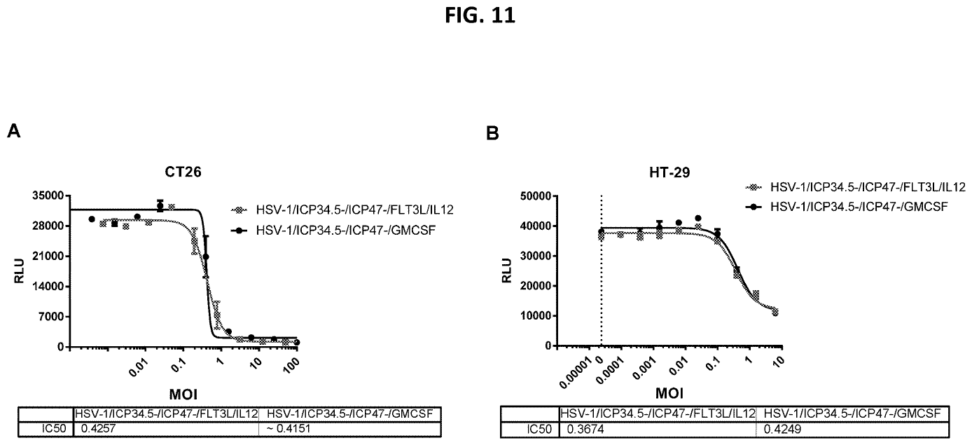

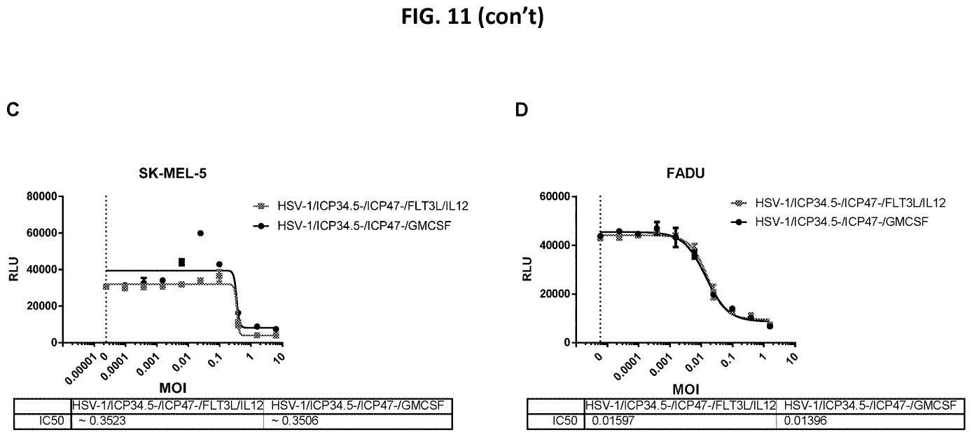

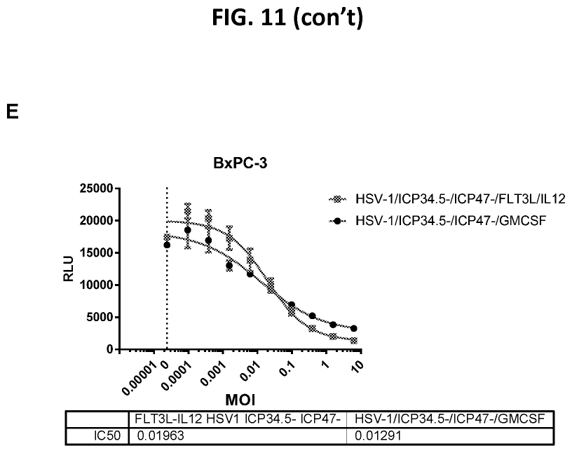

To confirm that the modifications introduced to the virus did not affect the ability of the virus to infect and lyse cells, in vitro killing assays were performed. A variety of cell lines of both mouse (CT26) and human (HT-29, SK-MEL-5, FADU, and BxPC3) origin were cultured with various multiplicities of infection (MOI) of viral particles (FIG. 11A-E). The results are discussed, below.

### Mouse Colorectal Cancer (CT26)

CT26 cells were plated in a 96-well plate at 6,000 cells per well and incubated overnight at 37° C. HSV-1/ICP34.5⁻/ICP47⁻/FLT3L/IL12 and HSV-1/ICP34.5⁻/ICP47⁻/GMCSF were serially diluted (4-fold, 10 wells) beginning at 100 MOI. After a 72-hour incubation, the number of cells left in each well was quantified using CellTiter-Glo Luminescent cell viability assay (Promega, Madison, WI).

### Human Cancer Cell Lines (HT-29, SK-MEL-5, FADU and BxPC-3)

Various human solid tumors cell lines (colorectal, melanoma, head and neck squamous carcinoma and pancreatic) were plated in a 96-well plate at 7,000-10,000 cells per well and incubated overnight at 37° C. HSV-1/ICP34.5⁻/ICP47⁻/FLT3L/IL12 and HSV-1/ICP34.5⁻/ICP47⁻/GMCSF were serially diluted (4-fold, 10 wells) beginning at 100 MOI. After a 72-hour incubation, the number of cells left in each well was quantified using CellTiter-Glo Luminescent cell viability assay (Promega #G7571, Madison, WI) on a SpectraMax M5 microplate reader (Molecular Devices Corporation).

HSV-1/ICP34.5⁻/ICP47⁻/FLT3L/IL12 was efficacious against all cancer cell lines tested. All cell lines tested had MOI IC₅₀ values below 1. FIG. 11 shows the degree of cell growth inhibition achieved by increasing concentrations of HSV-1/ICP34.5⁻/ICP47⁻/FLT3L/IL12 in each of the five cell lines, along with the MOI IC₅₀ values. These results demonstrate that treatment of colorectal, melanoma, head and neck and pancreatic cancer cell lines with HSV-1/ICP34.5⁻/ICP47⁻/FLT3L/IL12 results in strong inhibition of tumor cell growth with MOI IC₅₀ values that are similar to HSV-1/ICP34.5⁻/ICP47⁻/GMCSF.

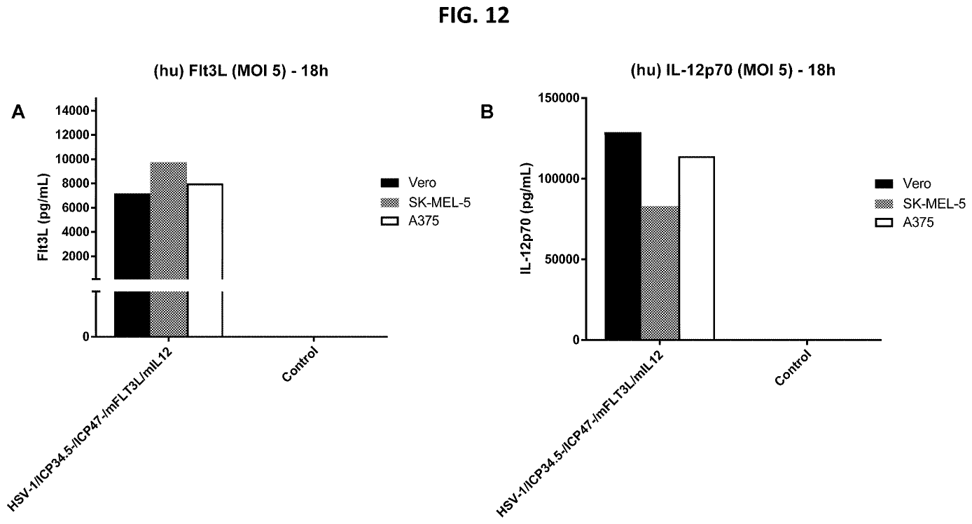

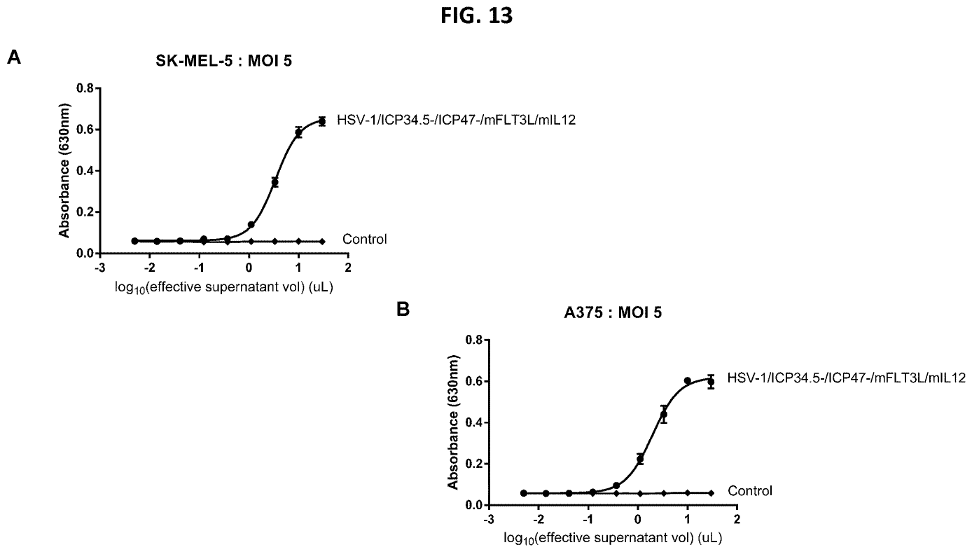

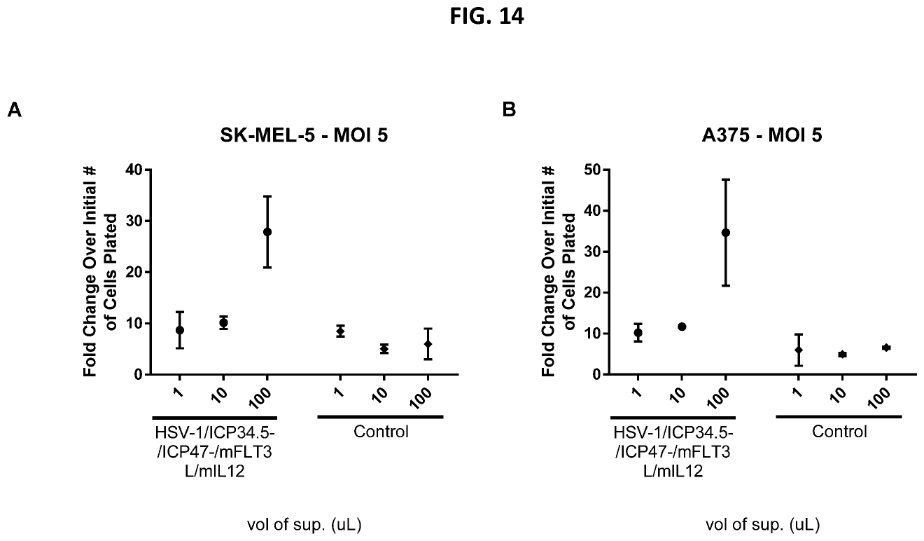

The production of bio-active FLT3L and IL12 in vitro as a result of HSV-1/ICP34.5⁻/ICP47⁻/FLT3L/IL12 infection was evaluated. The ELISA expression, IL12 reporter assay and FLT3L cell proliferation assay was repeated using supernatants from virally infected cells. Supernatants from the A375 and VERO cells used to confirm replication were screened as previously described. IL12p70 ELISA confirmed the expression of IL12 from all cell lines tested (VERO, A375, and SK-MEL-5) (FIG. 12A). In addition, the FLT3L ELISA demonstrated expression of FLT3L from all cell lines tested (FIG. 12B). Proof of IL12 bioactivity was established using the previously described IL12 reporter assay and BaF3 cell line proliferation assay. The virus infected cell supernatants showed active IL12 in a dose dependent fashion in both SK-MEL-5 (FIG. 13A) and A375 cells (FIG. 13B). Proof of FLT3L bioactivity was demonstrated using the BaF3 cell line stimulated with supernatants from either SK-MEL-5 (FIG. 14A) or A375 (FIG. 14B) cell lines.

In all cases examined, the supernatants from virus infected cells contained bioactive IL12 and FLT3L as expected based on the engineering specifications.

### Example 5: HSV-1/ICP34.5⁻/ICP47⁻/mFLT3L/mIL12 Virus is Capable of Producing Bio-Active FLT3L and IL12 In Vivo Upon Treatment of B Cell Lymphoma Tumor Bearing Animals (A20 Cell Line)

The expression of the dual cytokine payloads encoded by HSV-1/ICP34.5⁻/ICP47⁻/mFLT3L/mIL12 in the mouse A20 tumor model was evaluated.

A20 tumor cells (2×10⁶ cells) were injected subcutaneously in the right flanks of female Balb/c mice on day 0. Tumor volume (mm³) was measured using electronic calipers twice per week (Q2W). Once tumors reached an average of approximately 230 mm³, animals were randomized into 5 groups (4 mice per group) such that the average tumor volume and the variability of tumor volume at the beginning of treatment administration were uniform across treatment groups. Mice received a single intratumoral injection of HSV-1/ICP34.5⁻/ICP47⁻/mFLT3L/mIL12, HSV-1/ICP34.5⁻/ICP47⁻/mGMCSF, HSV-1/ICP34.5⁻/ICP47⁻/mFLT3L or HSV-1/ICP34.5⁻/ICP47⁻/mIL12 (each at 1×10⁶ PFU/dose), and then tumors and plasma were collected 16 hours later. mGM-CSF, mFLT3L and mIL12 levels were measured in tumor lysates and plasma from each treatment group using an MSD assay (mGM-CSF and mIL12 (mIL-12 nucleic acid shown in SEQ ID NO: 15; mIL-12 amino acid shown in SEQ ID NO: 16)) or R&D Quantikine ELISA (mFLT3L).

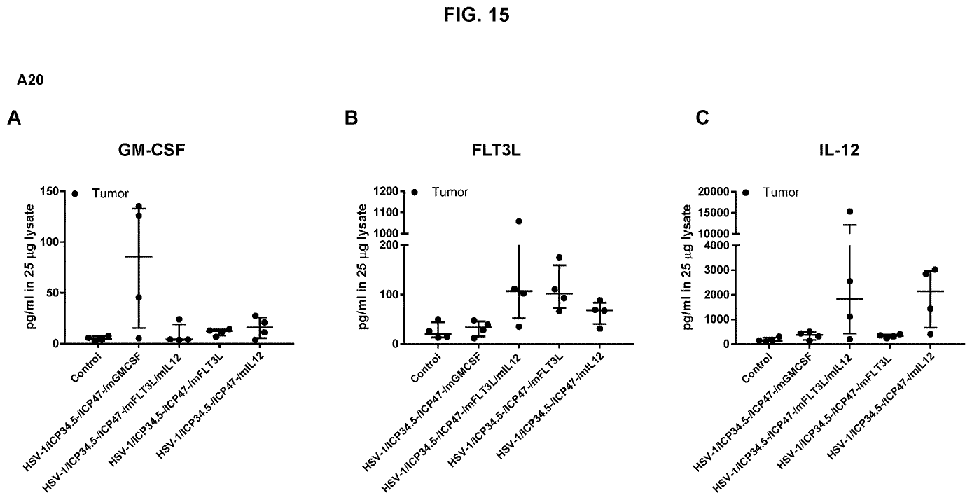

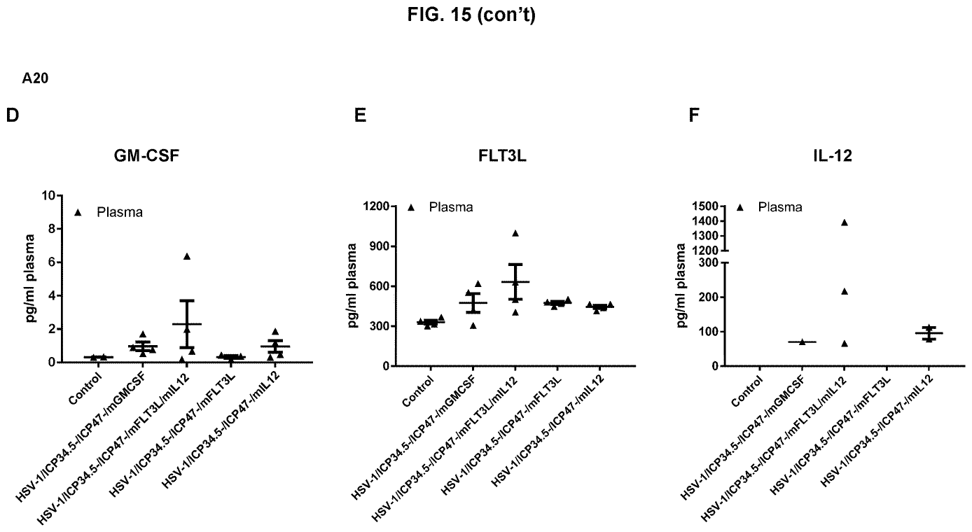

The results (FIG. 15) indicate that a single intratumoral dose of HSV-1/ICP34.5⁻/ICP47⁻/mFLT3L/mIL12 leads to expression of both mFLT3L and mIL12 in A20 tumor lysates and plasma at 16 hours.

### Example 6: HSV-1/ICP34.5⁻/ICP47⁻/mFLT3L/mIL12 Virus Produces Bio-Active FLT3L and IL12 In Vivo Upon Treatment of Melanoma Tumor Bearing Animals (B16F10 Cell Line)

The expression of the dual cytokine payloads encoded by HSV-1/ICP34.5⁻/ICP47⁻/mFLT3L/mIL12 in the mouse B16F10-mNectin1 tumor model was evaluated.

B16F10-mNectin1 tumor cells (3×10⁵ cells) were injected subcutaneously in the right flanks of female C57Bl/6 mice on day 0. Tumor volume (mm³) was measured using electronic calipers twice per week (Q2W). Once tumors reached an average of approximately 210 mm³, animals were randomized into 5 groups (4 mice per group) such that the average tumor volume and the variability of tumor volume at the beginning of treatment administration were uniform across treatment groups. Mice received a single intratumoral injection of HSV-1/ICP34.5⁻/ICP47⁻/mFLT3L/mIL12, HSV-1/ICP34.5⁻/ICP47⁻/mGMCSF, HSV-1/ICP34.5⁻/ICP47⁻/mFLT3L or HSV-1/ICP34.5⁻/ICP47⁻/mIL12 (each at 5×10⁶ PFU/dose), and then tumors and plasma were collected 16 hours later. mGM-CSF, mFLT3L and mIL12 levels were measured in tumor lysates and plasma from each treatment group using an MSD assay (mGM-CSF and mIL12) or R&D Quantikine ELISA (mFLT3L).

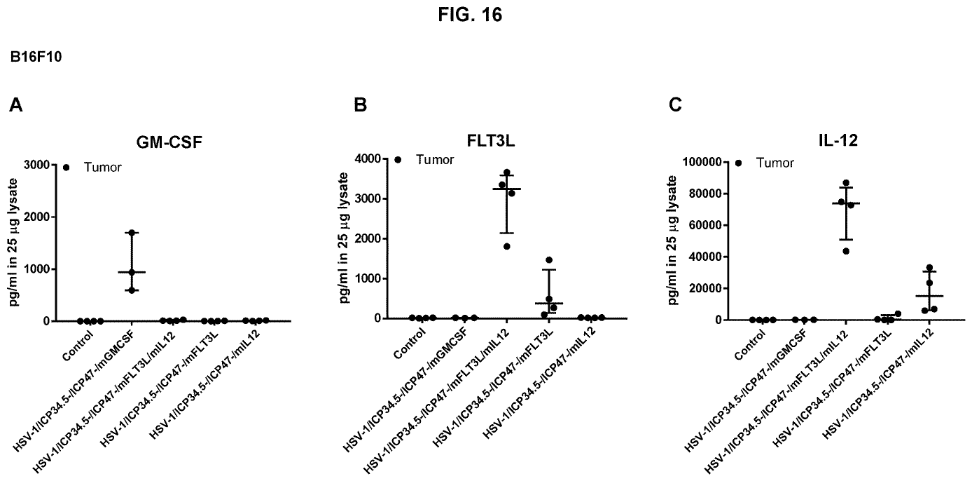

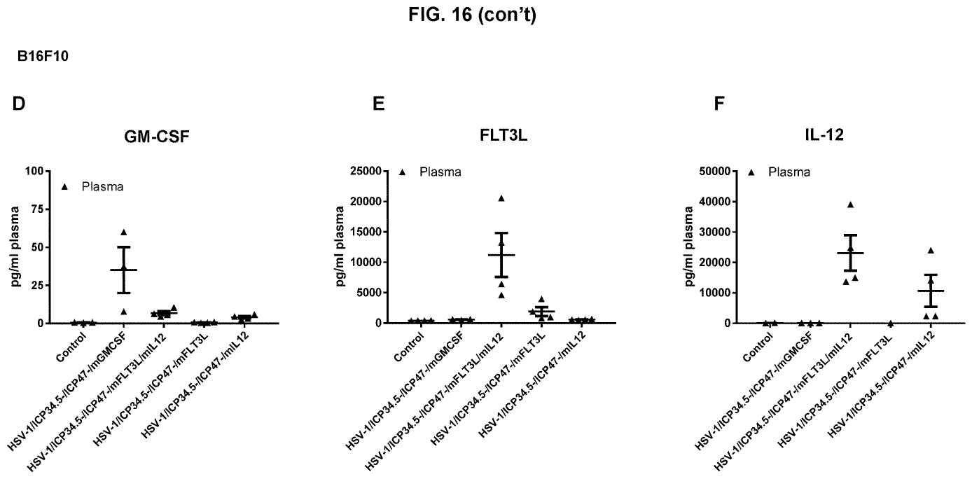

The results (FIG. 16) indicate that a single intratumoral dose of HSV-1/ICP34.5⁻/ICP47⁻/mFLT3L/mIL12 leads to expression of both mFLT3L and mIL12 in A20 tumor lysates and plasma at 16 hours.

### Example 7: HSV-1/ICP34.5⁻/ICP47⁻/mFLT3L/mIL12 Virus Elicits Systemic Anti-Tumor Immune Responses after Intra-Tumoral Injections In Vivo

The systemic anti-tumor T-cell responses elicited by treatment with HSV-1/ICP34.5⁻/ICP47⁻/mFLT3L/mIL12 was evaluated.

A20 tumor cells (2×10⁶ cells) were injected subcutaneously in the right and left flanks of female Balb/c mice on day 0. Tumor volume (mm³) was measured using electronic calipers twice per week (Q2W). Once tumors reached an average of approximately 100 mm³ (day 11), animals were randomized into 3 groups (12 mice per group) such that the average tumor volume (in both flanks) and the variability of tumor volume at the beginning of treatment administration were uniform across treatment groups. HSV-1/ICP34.5⁻/ICP47⁻/mFLT3L/mIL12 and HSV-1/ICP34.5⁻/ICP47⁻/mGMCSF (3×10⁴ PFU/dose) or formulation buffer control were administered intratumorally (on the right side of the animal) on study days 11, 14 and 17. The contralateral tumors (on the left side of the animal) received no injection. The study was terminated on day 21 and spleens were collected. Splenocytes were isolated from individual spleens and used in a whole-cell ELISpot assay (CTL, Shaker Heights, OH) to measure the number of T-cells secreting mIFN-γ when mixed with A20 tumor cells. Briefly, 7.5×10⁴ splenocytes were mixed with 1.5×10⁴ A20 tumor cells and incubated for 20 hours at 37° C. A CTLS6 Fluorospot analyzer (CTL, Shaker Heights, OH) was used to read the assay and enumerate the IFN-γ+ spots.

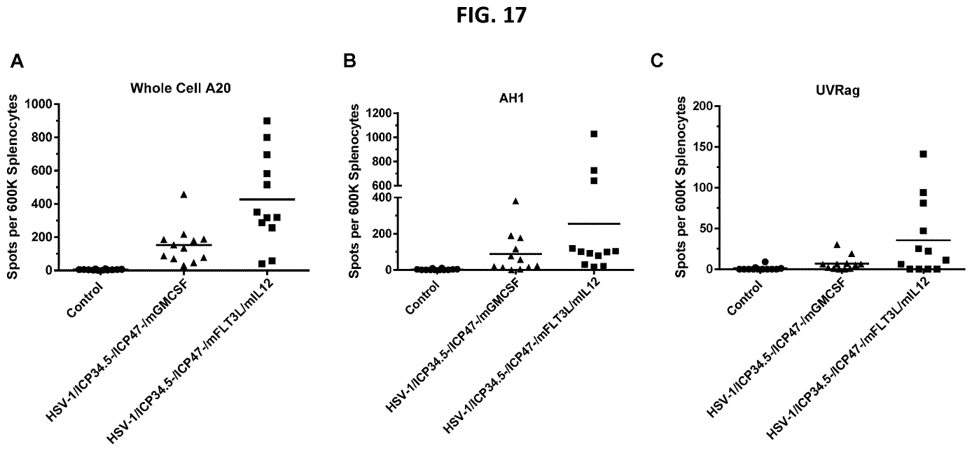

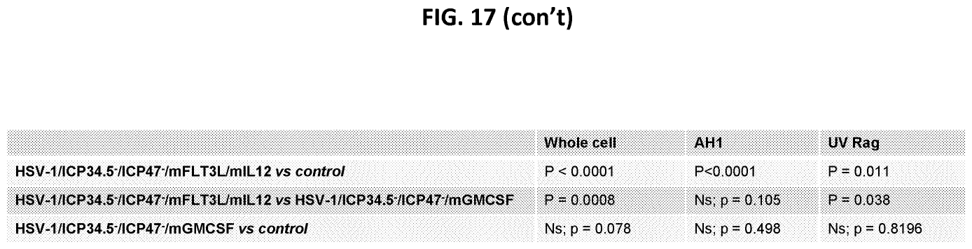

The results (FIG. 17A) indicate that treatment with HSV-1/ICP34.5⁻/ICP47⁻/mFLT3L/mIL12 led to a significantly increased systemic anti-A20 tumor activity compared to HSV-1/ICP34.5⁻/ICP47⁻/mGMCSF treatment (427 spots per 7.5×10⁴ splenocytes versus 152 spots, respectively; p=0.0008). In addition to whole tumor cells, the EliSpot was performed using an identified viral antigen associated with the A20 cell line, AH1 (FIG. 17B) and a neo-antigen mutation identified in the A20 cell line, UV Rag (FIG. 17C).

### Example 8: HSV-1/ICP34.5⁻/ICP47⁻/mFLT3L/mIL12 Elicits Anti-Tumor Efficacy in a Syngeneic Mouse B Cell Lymphoma Tumor Model (A20 Cells)

This study was designed to evaluate the tolerability and anti-tumor activity of HSV-1/ICP34.5⁻/ICP47⁻/mFLT3L/mIL12 and HSV-1/ICP34.5⁻/ICP47⁻/mGMCSF in a contralateral mouse A20 tumor model.

A20 tumor cells (2×10⁶ cells) were injected subcutaneously in the right and left flanks of female Balb/c mice on day 0. Tumor volume (mm³) was measured using electronic calipers twice per week (Q2W). Once tumors reached an average volume of approximately 100 mm³, animals were randomized into 6 groups (10 mice per group) such that the average tumor volume (in both flanks) and the variability of tumor volume at the beginning of treatment administration were uniform across treatment groups. HSV-1/ICP34.5⁻/ICP47⁻/mFLT3L/mIL12 and HSV-1/ICP34.5⁻/ICP47⁻/mGMCSF (3×10⁴ PFU/dose) or formulation buffer control were administered intratumorally (on the right side of the animal) every three days for three total injections. The contralateral tumors (on the left side of the animal) received no injection. Clinical signs, body weight changes, and survival (mice were removed from study when tumors reached 800 mm³) were measured 2 times weekly until study termination.

All animals survived through the experiment and showed no evidence of adverse health effects associated with treatment evidenced by body weight, and there were no noted adverse clinical signs identified on daily health monitoring examinations.

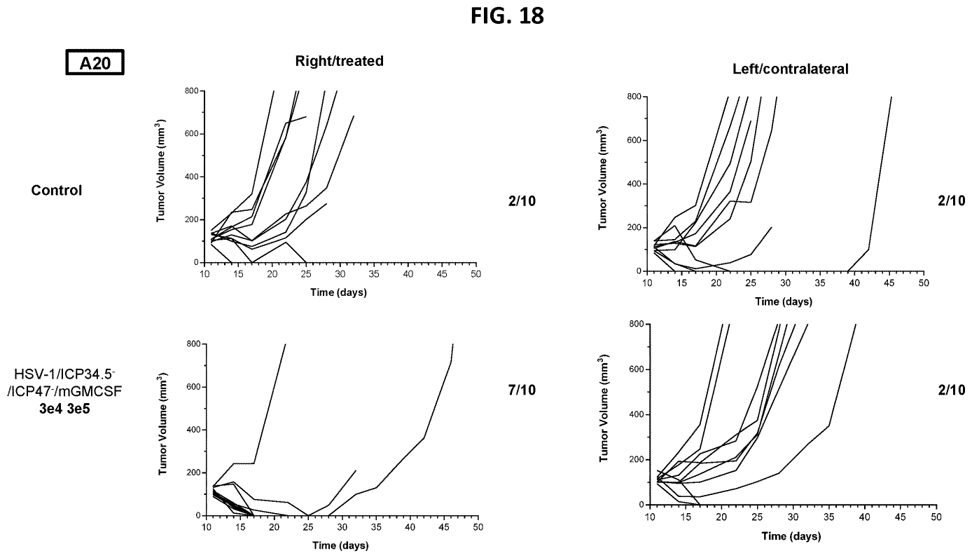

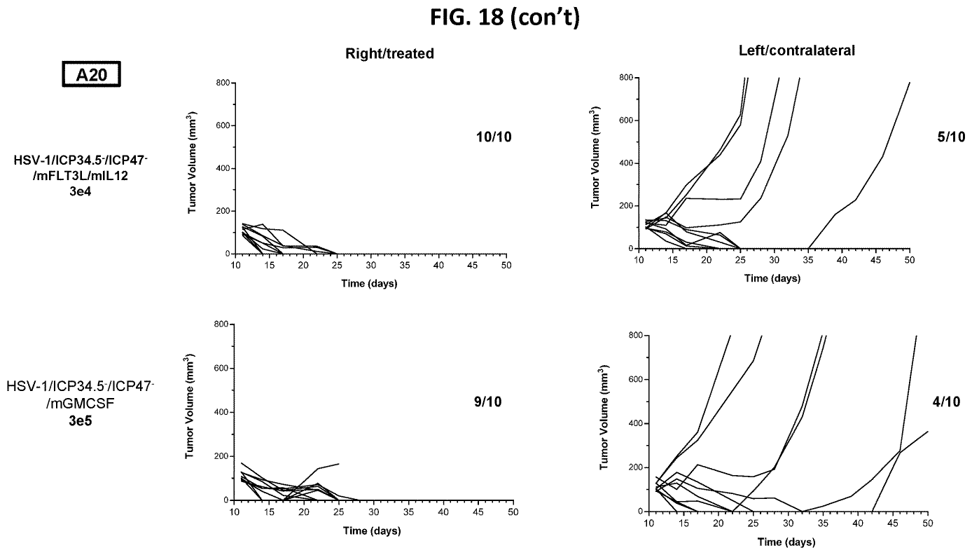

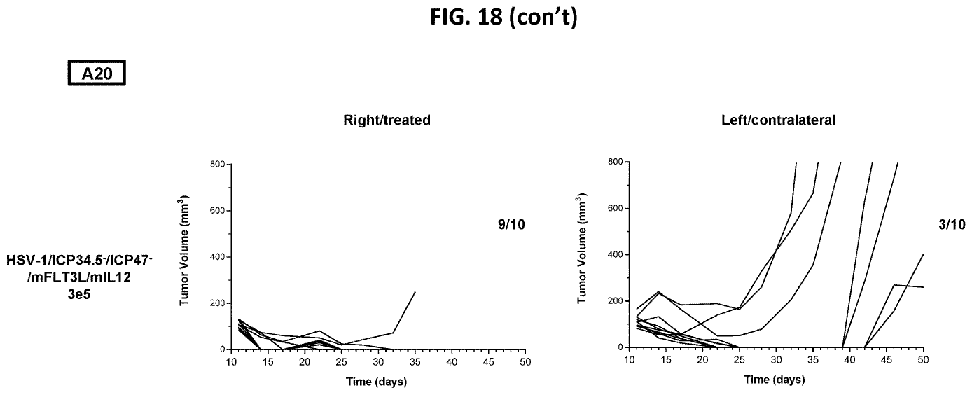

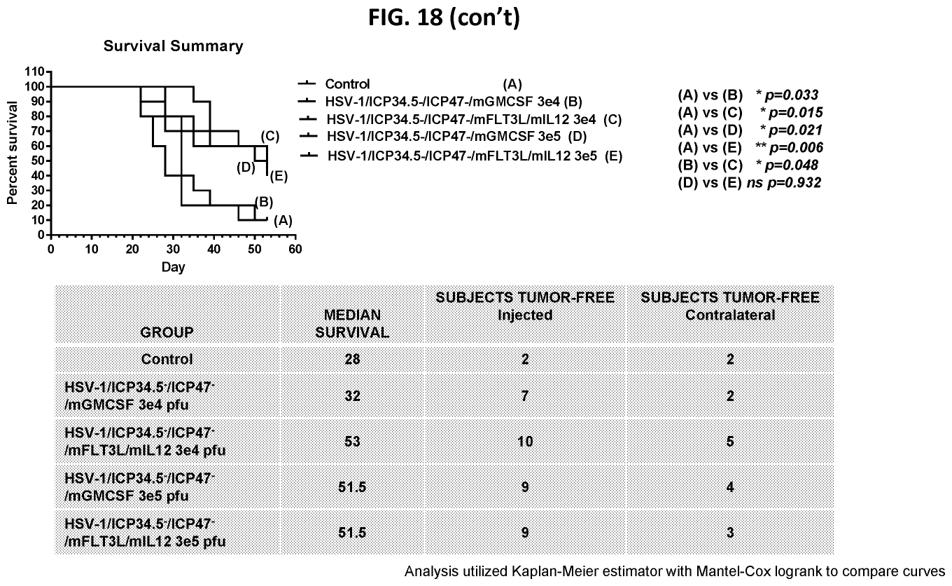

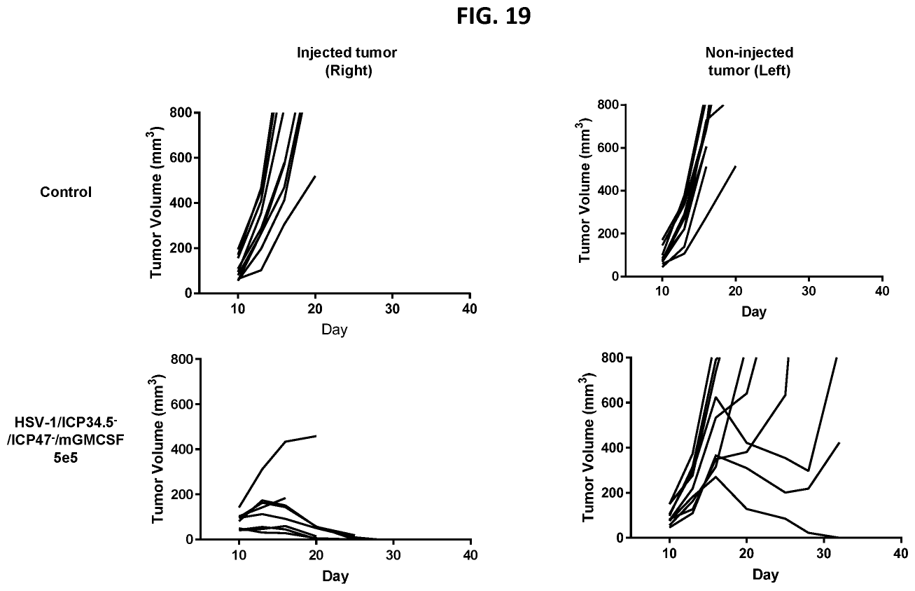

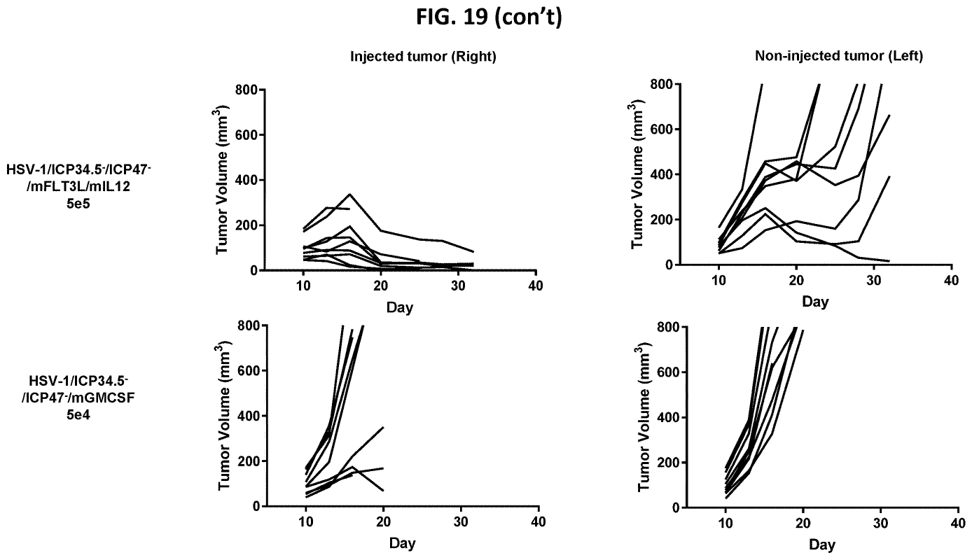

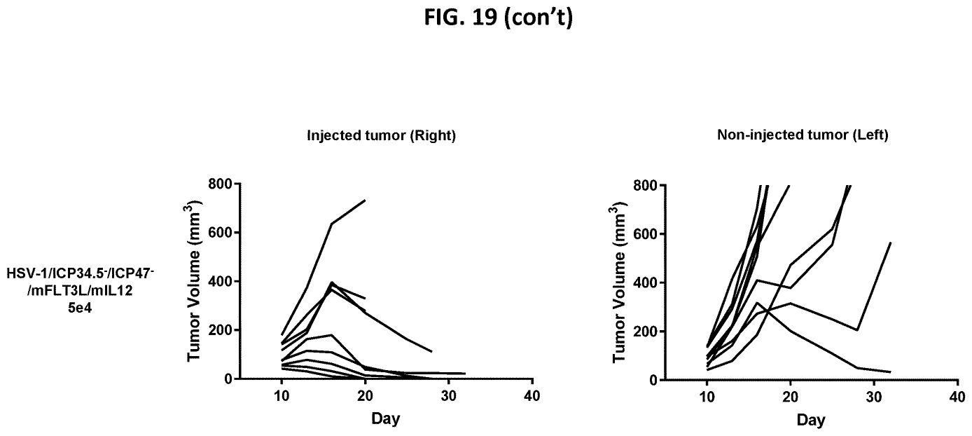

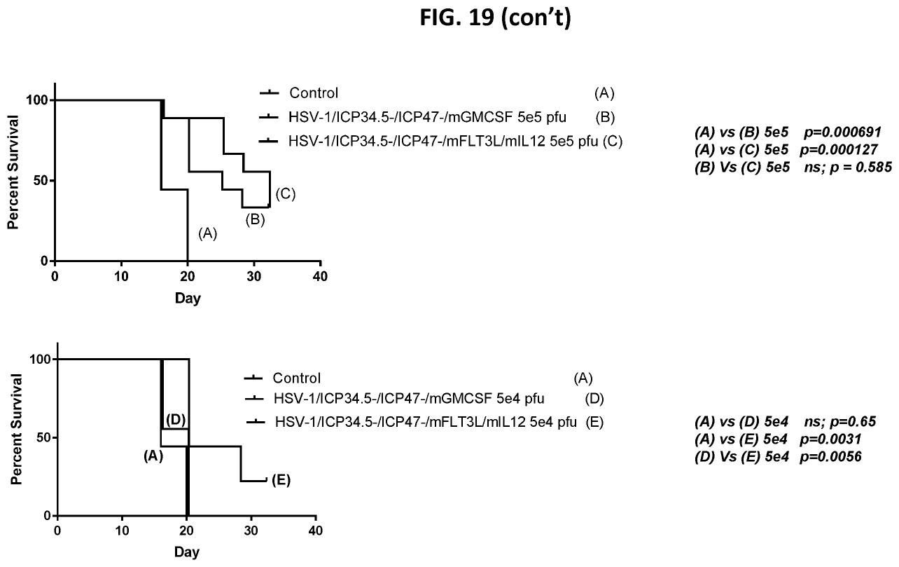

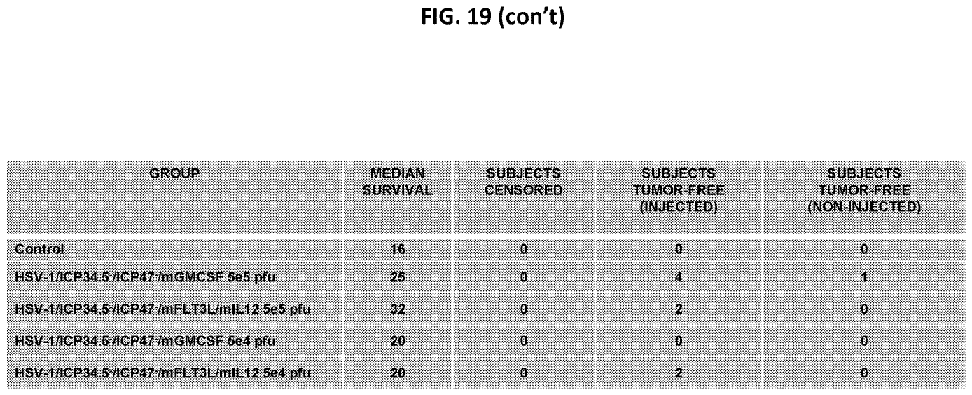

Tumor growth inhibition was observed in both treated (right side) and untreated (left side) tumors in both HSV-1/ICP34.5⁻/ICP47⁻/mFLT3L/mIL12 and HSV-1/ICP34.5⁻/ICP47⁻/mGMCSF treated groups in a dose dependent fashion (FIG. 18). However, there was an increase in complete responses (10/10 versus 7/10) in treated tumors and contralateral tumors (5/10 versus 2/10) in the HSV-1/ICP34.5⁻/ICP47⁻/mFLT3L/mIL12 treated animals compared to those treated with HSV-1/ICP34.5⁻/ICP47⁻/mGMCSF. Median survival was significantly increased in the HSV-1/ICP34.5⁻/ICP47⁻/mFLT3L/mIL12 treated group compared to HSV-1/ICP34.5⁻/ICP47⁻/mGMCSF (53 days versus 32 days, respectfully; p 0.048).

These data indicate that HSV-1/ICP34.5⁻/ICP47⁻/mFLT3L/mIL12 treatment led to improved contralateral tumor clearance and improved overall survival.

### Example 9: Study Evaluating HSV-1/ICP34.5⁻/ICP47⁻/mFLT3L/mIL12 and HSV-1/ICP34.5⁻/ICP47⁻/mGMCSF Efficacy in a Mouse Neuroblastoma (Neuro2A) Tumor Model

This study was designed to evaluate the tolerability and anti-tumor activity of HSV-1/ICP34.5⁻/ICP47⁻/mFLT3L/mIL12 and HSV-1/ICP34.5⁻/ICP47⁻/mGMCSF in a contralateral mouse Neuro2A tumor model

Neuro2A tumor cells (1×10⁶ cells) were injected subcutaneously in the right and left flanks of female Balb/c mice on day 0. Tumor volume (mm³) was measured using electronic calipers twice per week (Q2W). Once tumors reached an average volume of approximately 100 mm³, animals were randomized into groups (10 mice per group) such that the average tumor volume (in both flanks) and the variability of tumor volume at the beginning of treatment administration were uniform across treatment groups. HSV-1/ICP34.5⁻/ICP47⁻/mFLT3L/mIL12 and HSV-1/ICP34.5⁻/ICP47⁻/mGMCSF (5×10⁵ or 5×10⁴ PFU/dose) or formulation buffer control were administered intratumorally (on the right side of the animal) every three days for three total injections. The uninjected tumors (contralateral; on the left side of the animal) received no injection. Clinical signs, body weight changes, and survival (mice were removed from study when tumors reached 800 mm³) were measured 2 times weekly until study termination.

All animals survived through the experiment and showed no evidence of adverse health effects associated with treatment evidenced by body weight, and there were no noted adverse clinical signs identified on daily health monitoring examinations.

At 5e5 PFU per dose, both the HSV-1/ICP34.5⁻/ICP47⁻/mFLT3L/mIL12 treated group and the HSV-1/ICP34.5⁻/ICP47⁻/mGMCSF treated group were statistically significant compared to control treated animals. At 5e4 PFU per dose, the overall survival of HSV-1/ICP34.5⁻/ICP47⁻/mFLT3L/mIL12 treated group compared to HSV-1/ICP34.5⁻/ICP47⁻/mGMCSF was increased (although the median survival for both groups was 20 days; p=0.0056).

These data indicate that HSV-1/ICP34.5⁻/ICP47⁻/mFLT3L/mIL12 treatment led to an improved contralateral tumor clearance and improved overall survival.

### Example 10: Study Evaluating HSV-1/ICP34.5⁻/ICP47⁻/mFLT3L/mIL12 and HSV-1/ICP34.5⁻/ICP47⁻/mGMCSF Efficacy in a Mouse Neuroblastoma (CT26) Tumor Model

This study was designed to evaluate the tolerability and anti-tumor activity of HSV-1/ICP34.5⁻/ICP47⁻/mFLT3L/mIL12 and HSV-1/ICP34.5⁻/ICP47⁻/mGMCSF in a contralateral mouse CT26 (also known as colon26) tumor model.

CT26 tumor cells (3×10⁵ cells) were injected subcutaneously in the right and left flanks of female Balb/c mice on day 0. Tumor volume (mm³) was measured using electronic calipers twice per week (Q2W). Once tumors reached an average volume of approximately 100 mm³, animals were randomized into groups (10 mice per group) such that the average tumor volume (in both flanks) and the variability of tumor volume at the beginning of treatment administration were uniform across treatment groups. HSV-1/ICP34.5⁻/ICP47⁻/mFLT3L/mIL12, HSV-1/ICP34.5⁻/ICP47⁻/mGMCSF (5×10⁶ PFU/dose), or formulation buffer control were administered intratumorally (on the right side of the animal) every three days for three total injections. The uninjected tumors (contralateral; on the left side of the animal) received no injection. Clinical signs, body weight changes, and survival (mice were removed from study when tumors reached 800 mm³) were measured 2 times weekly until study termination.

All animals survived through the experiment and showed no evidence of adverse health effects associated with treatment evidenced by body weight, and there were no noted adverse clinical signs identified on daily health monitoring examinations.

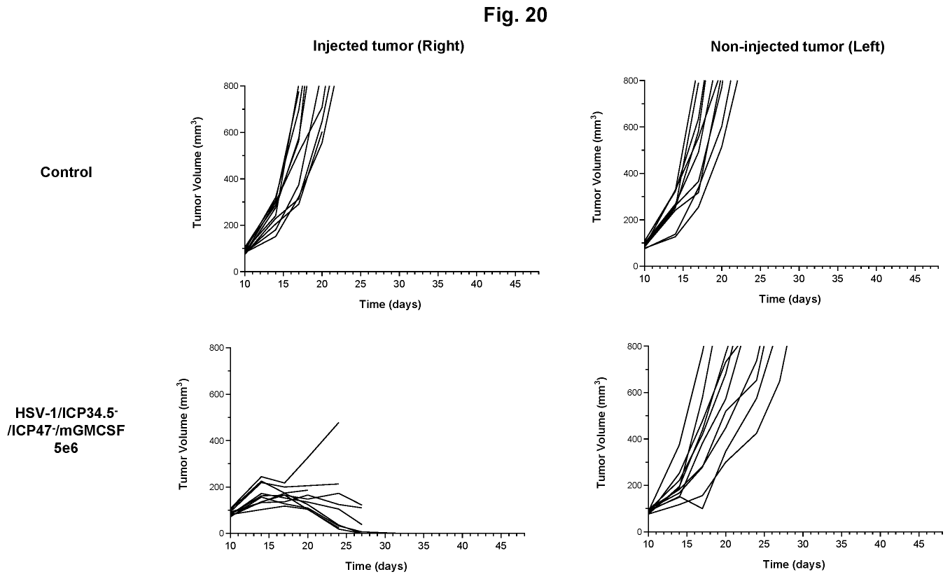

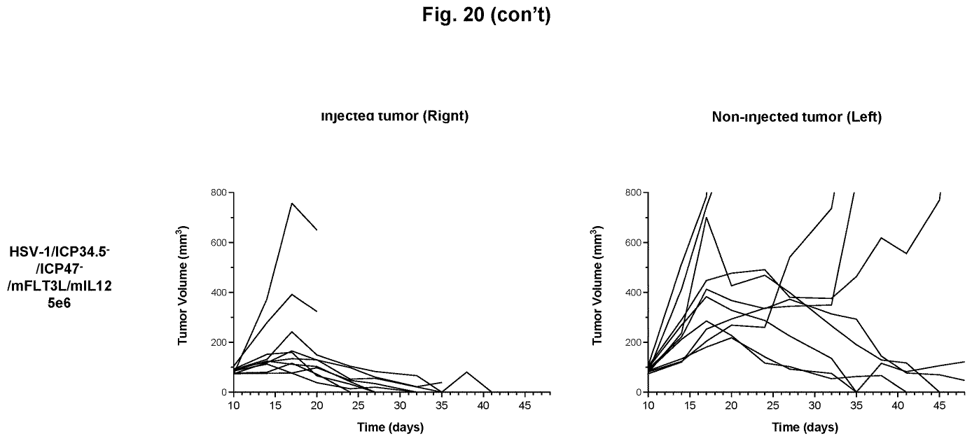

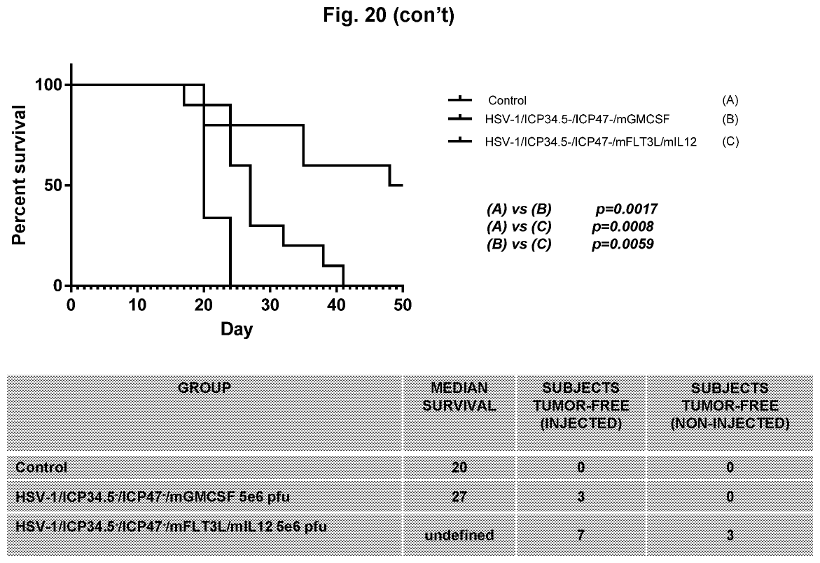

At 5×10⁶ PFU per dose, the survival of both the HSV-1/ICP34.5⁻/ICP47⁻/mFLT3L/mIL12 treated group and the HSV-1/ICP34.5⁻/ICP47⁻/mGMCSF treated group was significantly increased as compared to control treated animals (control vs HSV-1/ICP34.5⁻/ICP47⁻/mGMCSF; p=0.0017 and control vs HSV-1/ICP34.5⁻/ICP47⁻/mFLT3L/mIL12; p=0.0008). Additionally, the overall survival of HSV-1/ICP34.5⁻/ICP47⁻/mFLT3L/mIL12 treated group compared to HSV-1/ICP34.5⁻/ICP47⁻/mGMCSF was increased (median survival not defined for HSV-1/ICP34.5⁻/ICP47⁻/mFLT3L/mIL12 as compared to 27 days for HSV-1/ICP34.5⁻/ICP47⁻/mGMCSF; p=0.0059). See FIG. 20.

These data indicate that HSV-1/ICP34.5⁻/ICP47⁻/mFLT3L/mIL12 treatment led to an improved contralateral tumor clearance and improved overall survival as compared to either control treatment or HSV-1/ICP34.5⁻/ICP47⁻/mGMCSF treatment.

### Example 11: Study Evaluating HSV-1/ICP34.5⁻/ICP47⁻/mFLT3L/mIL12 in Combination with Checkpoint Blockade (Anti-PD1 mAb) Efficacy in a Mouse Colorectal (MC38) Tumor Model

This study was designed to evaluate the tolerability and anti-tumor activity of HSV-1/ICP34.5⁻/ICP47⁻/mFLT3L/mIL12 alone or in combination with anti-programmed cell death protein 1 (PD1) monoclonal antibody (mAb) in a contralateral mouse MC38 tumor model.

MC38 tumor cells (3×10⁵ cells) were injected subcutaneously in the right and left flanks of female C57BL/6 mice on day 0. Tumor volume (mm³) was measured using electronic calipers twice per week (Q2W). Once tumors reached an average volume of approximately 100 mm³, animals were randomized into groups (10 mice per group) such that the average tumor volume (in both flanks) and the variability of tumor volume at the beginning of treatment administration were uniform across treatment groups. HSV-1/ICP34.5⁻/ICP47⁻/mFLT3L/mIL12 (5×10⁶ PFU/dose) or formulation buffer control were administered intratumorally (on the right side of the animal) every three days for three total injections. The uninjected tumors (contralateral; on the left side of the animal) received no injection. Anti-PD1 monoclonal antibody (200 μg/dose) was administered by intraperitoneal injection on the same schedule (every three days for three total injections). Clinical signs, body weight changes, and survival (mice were removed from study when tumors reached 800 mm³) were measured 2 times weekly until study termination.

All animals survived through the experiment and showed no evidence of adverse health effects associated with treatment evidenced by body weight, and there were no noted adverse clinical signs identified on daily health monitoring examinations.

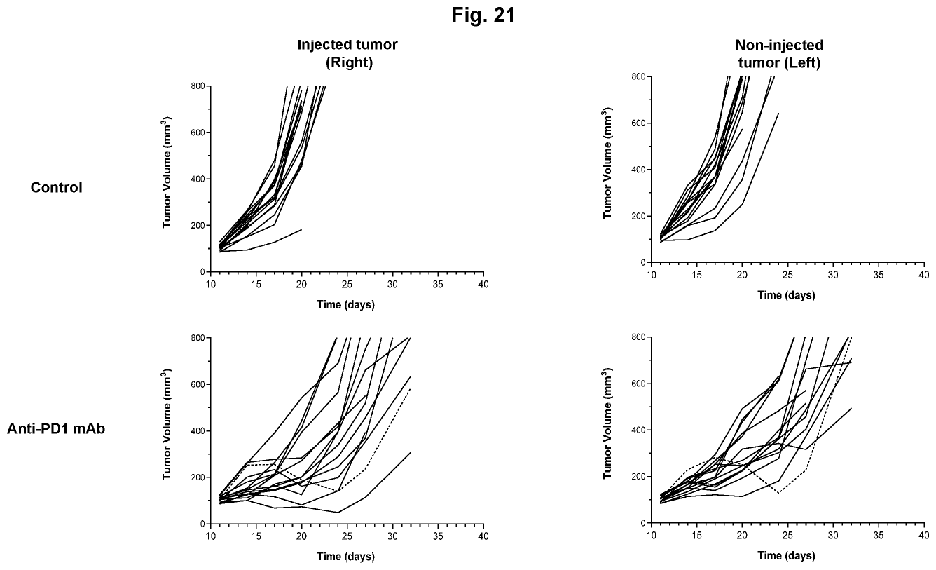

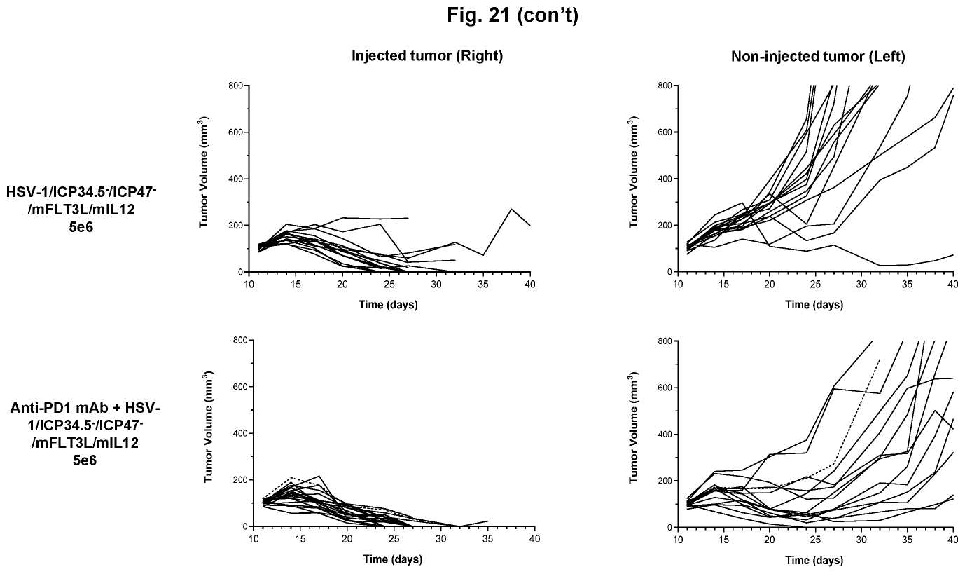

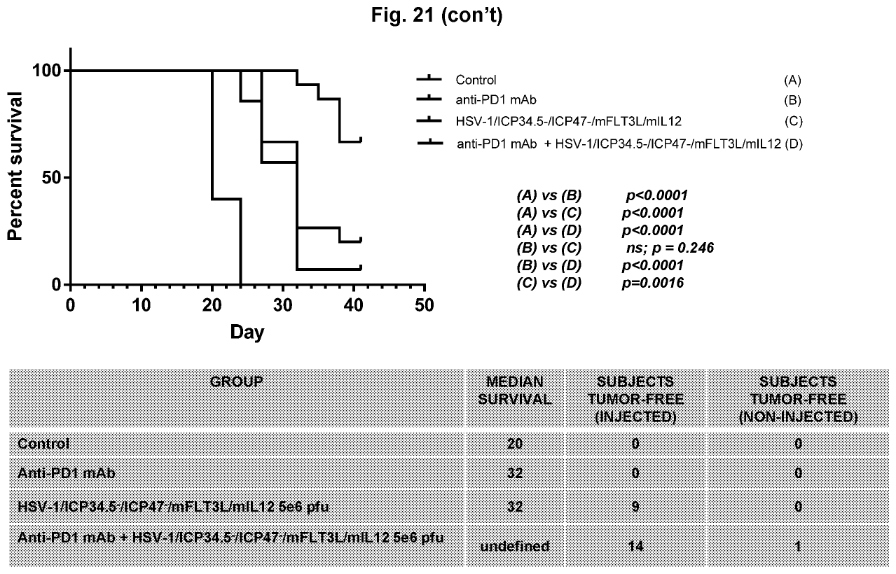

Both single treatments, anti-PD1 mAb alone, and 5×10⁶ PFU HSV-1/ICP34.5⁻/ICP47⁻/mFLT3L/mIL12 alone, demonstrated significantly increased survival as compared to control treated animals (p<0.0001 for each comparison respectively). Survival of anti-PD1 mAb alone treated animals was not statistically significant as compared to 5×10⁶ PFU HSV-1/ICP34.5⁻/ICP47⁻/mFLT3L/mIL12 alone (p=0.246). The combination of both treatments, anti-PD1 mAb plus 5×10⁶ PFU HSV-1/ICP34.5⁻/ICP47⁻/mFLT3L/mIL12, demonstrated significantly increased survival as compared to all other treatment groups (p=0.0016 as compared to 5×10⁶ PFU HSV-1/ICP34.5⁻/ICP47⁻/mFLT3L/mIL12 alone, p<0.0001 as compared to anti-PD1 mAb alone, and p<0.0001 as compared to control treatment). See FIG. 21.

These data indicate that while either HSV-1/ICP34.5⁻/ICP47⁻/mFLT3L/mIL12 or anti-PD1 mAb treatment alone led to a significant improvement in overall survival as compared to control treatment, the combination of both treatments resulted in a significantly improved overall survival as compared to either treatment alone.

### Example 12: Study Evaluating Kinetics of Cytokine Expression by HSV-1/ICP34.5⁻/ICP47⁻/mFLT3L/mIL12 in a Mouse Colorectal (CT26) Tumor Model

This study was designed to evaluate the kinetics of cytokine expression by HSV-1/ICP34.5⁻/ICP47⁻/mFLT3L/mIL12 when injected in a mouse CT26 tumor model.

CT26 tumor cells (3×10⁵ cells) were injected subcutaneously in the right flank of female BALB/c mice on day 0. Tumor volume (mm³) was measured using electronic calipers twice per week (Q2W). Once tumors reached an average volume of approximately 100 mm³, animals were randomized into groups (5 mice per group for control, 25 mice per group for HSV-1/ICP34.5⁻/ICP47⁻, and 25 mice per group for HSV-1/ICP34.5⁻/ICP47⁻/mFLT3L/mIL12). The average tumor volume and the variability of tumor volume at the beginning of treatment administration were uniform across treatment groups. HSV-1/ICP34.5⁻/ICP47⁻ (5×10⁶ PFU/dose of virus; virus not containing a cytokine payload), HSV-1/ICP34.5⁻/ICP47⁻/mFLT3L/mIL12 (5×10⁶ PFU/dose of virus), and formulation buffer control were each administered intratumorally every three days for three total injections. Clinical signs and body weight changes were measured 2 times weekly until study termination. 5 mice per each virus treated group were euthanized at 4, 24, 72, 168 and 240 hours post administration of virus. 5 mice in the control treated group were taken down immediately after formulation buffer control injection. Blood was isolated and prepared as serum, tumors were excised from the animal and prepared as a protein lysate.

All animals survived through the experiment and showed no evidence of adverse health effects associated with treatment evidenced by body weight, and there were no noted adverse clinical signs identified on daily health monitoring examinations.

The serum and tumor protein lysates were analyzed for the presence of mouse FLT3L and IL-12, which are the two cytokines encoded by the virus HSV-1/ICP34.5⁻/ICP47⁻/mFLT3L/mIL12. Virus without a cytokine (HSV-1/ICP34.5⁻/ICP47⁻) was used to control for endogenous cytokine expression.

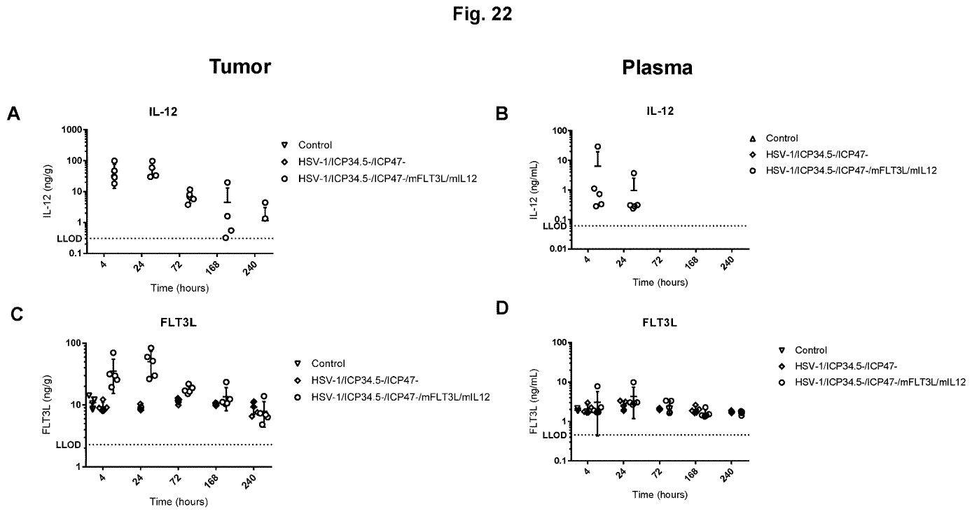

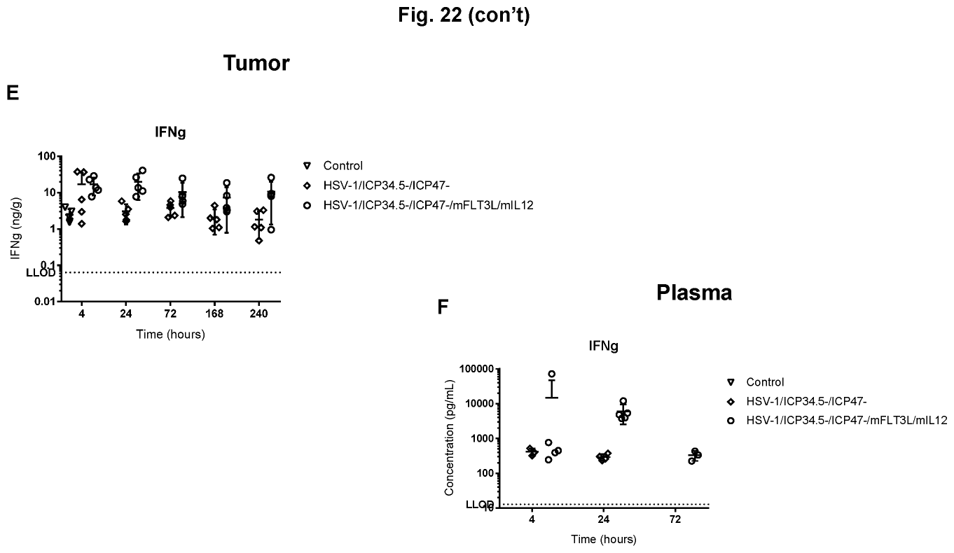

In the tumor lysate, all animals injected with HSV-1/ICP34.5⁻/ICP47⁻/mFLT3L/mIL12 showed expression of IL-12 in the tumor lysate out to day 7 (168 hours) post injection. 2 of 5 animals showed expression of IL-12 at day 10 (240 hours) post injection (FIG. 22A). All animals injected with either control or HSV-1/ICP34.5⁻/ICP47⁻ virus had levels of IL-12 that were below the lower limit of detection (LLOD). In the plasma, IL-12 was detected in all 5 animals injected with HSV-1/ICP34.5⁻/ICP47⁻/mFLT3L/mIL12 at 4 hours post injection. At 24 hours post injection, 4 of 5 animals injected with HSV-1/ICP34.5⁻/ICP47⁻/mFLT3L/mIL12 had detectable IL-12. All time points sampled after 24 hours were below the LLOD (FIG. 22B).

In the tumor lysate, all animals injected with HSV-1/ICP34.5⁻/ICP47⁻/mFLT3L/mIL12 showed a statistically significant increase in expression of FLT3L in the tumor lysate out to day 3 (72 hours) post injection (4 hour HSV-1/ICP34.5⁻/ICP47⁻ vs HSV-1/ICP34.5⁻/ICP47⁻/mFLT3L/mIL12, p=0.0197; 24 hour HSV-1/ICP34.5⁻/ICP47⁻ vs HSV-1/ICP34.5⁻/ICP47⁻/mFLT3L/mIL12, p=0.0043, 72 hour HSV-1/ICP34.5⁻/ICP47⁻ vs HSV-1/ICP34.5⁻/ICP47⁻/mFLT3L/mIL12, p=0.0012; 168 hour HSV-1/ICP34.5⁻/ICP47⁻ vs HSV-1/ICP34.5⁻/ICP47⁻/mFLT3L/mIL12, p=0.2281; 240 hour HSV-1/ICP34.5⁻/ICP47⁻ vs HSV-1/ICP34.5⁻/ICP47⁻/mFLT3L/mIL12, p=0.4890; FIG. 22C). In the plasma, FLT3L was detectable in all samples from all mice in all groups. There was no statistically significant difference between any groups at any timepoint (FIG. 22D).

In the tumor lysate, only animals injected with HSV-1/ICP34.5⁻/ICP47⁻ and HSV-1/ICP34.5⁻/ICP47⁻/mFLT3L/mIL12 showed significantly increased expression of IFN-γ in the tumor lysate as compared to control at 4 hours post injection (p=0.0057). 24, 72, 168 and 240 hours post injection, there was no detectable IFN-γ in the control treated tumors. 24 hours post injection, animals that received HSV-1/ICP34.5⁻/ICP47⁻/mFLT3L/mIL12 showed significantly elevated IFN-γ levels as compared to HSV-1/ICP34.5⁻/ICP47⁻ (p=0.0253). At 72, 168, and 240 hours post injection, the levels of IFN-γ in the HSV-1/ICP34.5⁻/ICP47⁻/mFLT3L/mIL12 trended higher than HSV-1/ICP34.5⁻/ICP47⁻ but failed to achieve statistical significance (p=0.2306, 0.1155, and p=0.0693; respectively; FIG. 22E). Sustained IFN-γ production at 24 hours post injection is consistent with the production of IL-12 and should prime an enhanced anti-tumor immune response. In the plasma, no IFN-γ was detected in animals treated with control injection. In animals treated with HSV-1/ICP34.5⁻/ICP47⁻/mFLT3L/mIL12 and HSV-1/ICP34.5⁻/ICP47⁻, there was no statistically significant difference in plasma IFN-γ at 4 hours post injection (p=0.4803), a significant increase at 24 hours post injection (p=0.0140), and IFN-γ was detected in HSV-1/ICP34.5⁻/ICP47⁻/mFLT3L/mIL12 at 72 hours. All other timepoints and conditions were below the lower limit of detection (LLOD) for the assay (FIG. 22F).

### Example 13: Study Evaluating the Ability of HSV-1/ICP34.5⁻/ICP47⁻/mFLT3L/mIL12 to Generate an Anti-Tumor T Cell Response

This study evaluated the anti-tumor immune response generated by the injection of HSV-1/ICP34.5⁻/ICP47⁻/mFLT3L/mIL12 in a contralateral mouse MC38 tumor model.

MC38 tumor cells (3×10⁵ cells) were injected subcutaneously in the right and left flanks of female C57BL/6 mice on day 0. Tumor volume (mm³) was measured using electronic calipers twice per week (Q2W). Once tumors reached an average volume of approximately 100 mm³, animals were randomized into groups (12 mice per group) such that the average tumor volume (in both flanks) and the variability of tumor volume at the beginning of treatment administration were uniform across treatment groups. HSV-1/ICP34.5⁻/ICP47⁻/mFLT3L/mIL12 (5×10⁶ PFU/dose) or formulation buffer control were administered intratumorally (on the right side of the animal) every three days for three total injections. The uninjected tumors (contralateral; on the left side of the animal) received no injection. Anti-PD1 monoclonal antibody (200 μg/dose) was administered by intraperitoneal injection on the same schedule (every three days for three total injections). Clinical signs, body weight changes, and tumor volumes were measured 2 times weekly until study termination on day 21.

All animals survived through the experiment and showed no evidence of adverse health effects associated with treatment evidenced by body weight, and there were no noted adverse clinical signs identified on daily health monitoring examinations.

The mice were euthanized on day 21, spleens were excised and IFN-γ ELISpot assays (peptide restimulation and whole cell) were performed on single cell suspensions of splenocytes. For peptide restimulation assays, 5×10⁵ splenocytes were plated and stimulated overnight with single 9-mer peptides (representing either MC38 neoantigens or viral-derived tumor antigens) at a final concentration of 1 μM. Whole cell assays were set up by plating 1.25×10⁵ splenocytes with 1.25×10⁴ MC38 cells. In each assay, the enumeration of spots indicates the total number of IFN-γ expressing immune cells.

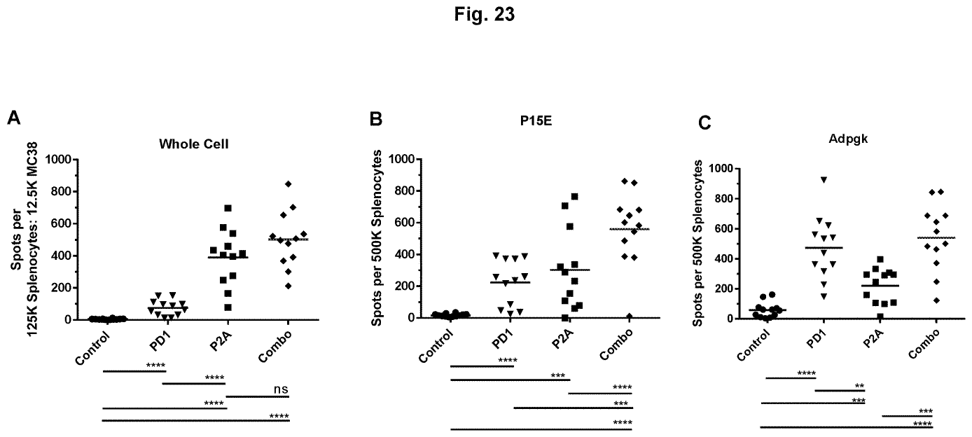

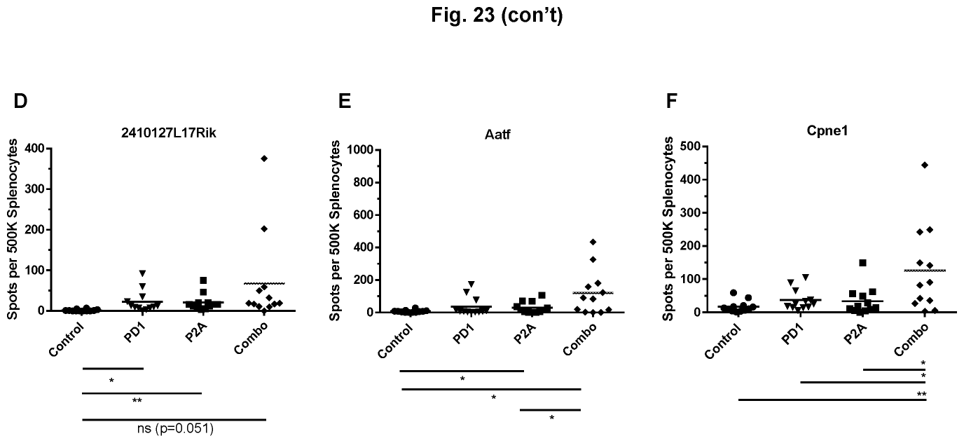

In the peptide restimulation assay, treatment with HSV-1/ICP34.5⁻/ICP47⁻/mFLT3L/mIL12 alone led to a significant increase in immune reactivity to MC38 tumor cells; in the whole cell assay, treatment with HSV-1/ICP34.5⁻/ICP47⁻/mFLT3L/mIL12 led to a significant increase in anti-MC38 activity compared to both control and anti-PD1 treated animals (p<0.0001 for both; FIG. 23A). Immune reactivity to viral-derived tumor antigen P15E was also significantly increased in HSV-1/ICP34.5⁻/ICP47⁻/mFLT3L/mIL12 treated as compared to control animals (p=0.0008; FIG. 23B).

MC38 contains several genomic mutations that result in neoantigens. Immune reactivity to these tumor specific mutations was quantitated. In HSV-1/ICP34.5⁻/ICP47⁻/mFLT3L/mIL12 treated animals, reactivity to Adpgk (FIG. 23C), 2410127L17Rik (FIG. 23D), and Aatf (FIG. 23E) was significantly increased as compared to control treated mice (p=0.003, p=0.0416 and p=0.0035, respectively). In addition, the combination of HSV-1/ICP34.5⁻/ICP47⁻/mFLT3L/mIL12 and anti-PD1 blockade led to a significant increase in immune reactivity to Adpgk (p=0.002), Aatf (p=0.040), Cpne1 (p=0.030), and P15E (p=0.0008) compared to HSV-1/ICP34.5⁻/ICP47⁻/mFLT3L/mIL12 treatment alone. These data indicate that HSV-1/ICP34.5⁻/ICP47⁻/mFLT3L/mIL12 treatment can increase the anti-tumor immune response in the MC38 tumor model. This increase can be further enhanced by the addition of anti-PD1. The generation of a systemic anti-tumor response and its enhancement by checkpoint blockade should contribute to anti-tumor immunity against both injected and uninjected lesions, as demonstrated in efficacy studies herein.

### Example 14: Study Evaluating HSV-1/ICP34.5⁻/ICP47⁻/mFLT3L/mIL12 in Combination with 4-1BB Agonist mAb Efficacy in a Mouse Colorectal (MC38) Tumor Model

This study evaluated the tolerability and anti-tumor activity of HSV-1/ICP34.5⁻/ICP47⁻/mFLT3L/mIL12 alone or in combination with an agonistic antibody targeted 4-1BB (aka CD137) in a contralateral mouse MC38 tumor model.

MC38 tumor cells (3×10⁵ cells) were injected subcutaneously in the right and left flanks of female C57BL/6 mice on day 0. Tumor volume (mm³) was measured using electronic calipers twice per week (Q2W). Once tumors reached an average volume of approximately 100 mm³, animals were randomized into groups (10 mice per group) such that the average tumor volume (in both flanks) and the variability of tumor volume at the beginning of treatment administration were uniform across treatment groups. HSV-1/ICP34.5⁻/ICP47⁻/mFLT3L/mIL12 (5×10⁶ PFU/dose) or formulation buffer control were administered intratumorally (on the right side of the animal) every three days for three total injections. The uninjected tumors (contralateral; on the left side of the animal) received no injection. Anti-4-1BB monoclonal antibody (150 μg/dose) was administered by intraperitoneal injection on the same schedule (every three days for three total injections). Clinical signs, body weight changes, and survival (mice were removed from study when tumors reached 800 mm³) were measured 2 times weekly until study termination.

All animals survived through the experiment and showed no evidence of adverse health effects associated with treatment evidenced by body weight, and there were no noted adverse clinical signs identified on daily health monitoring examinations.

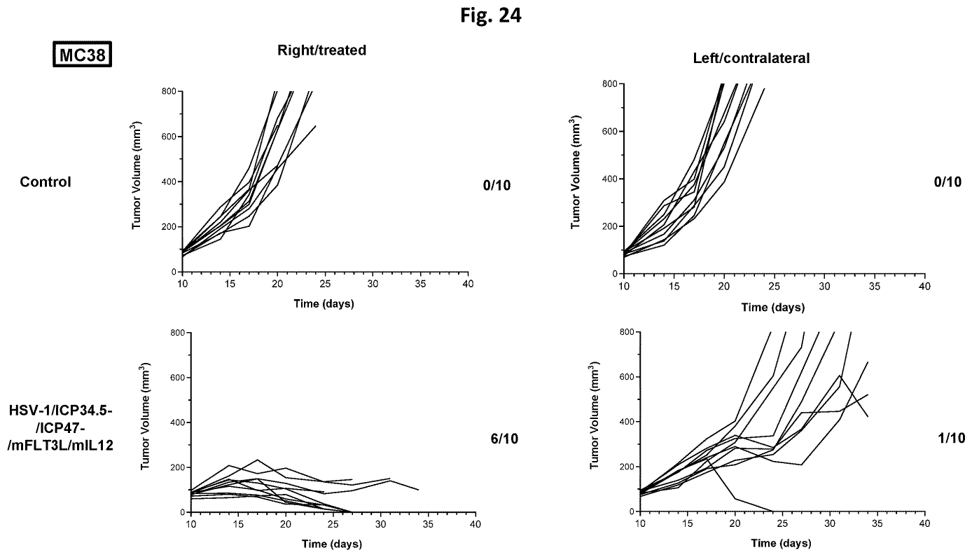

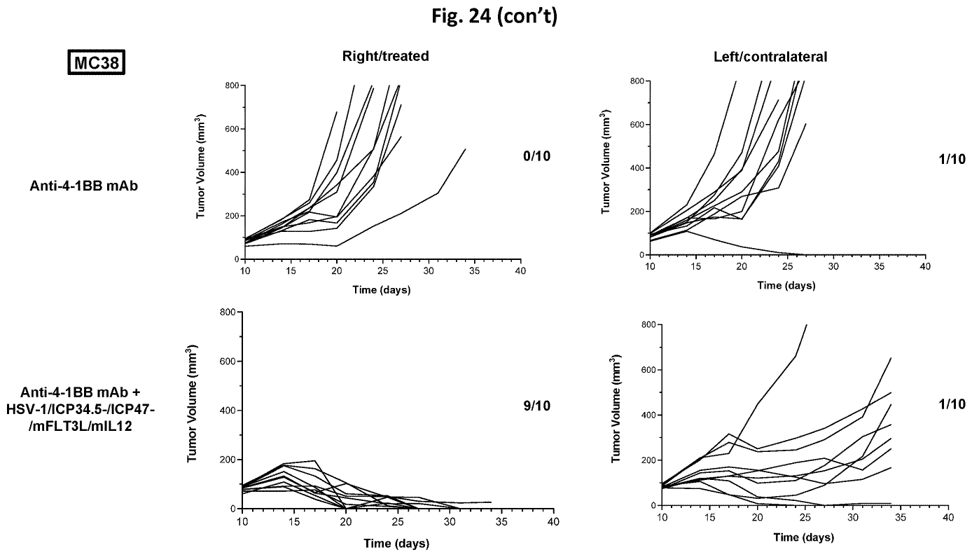

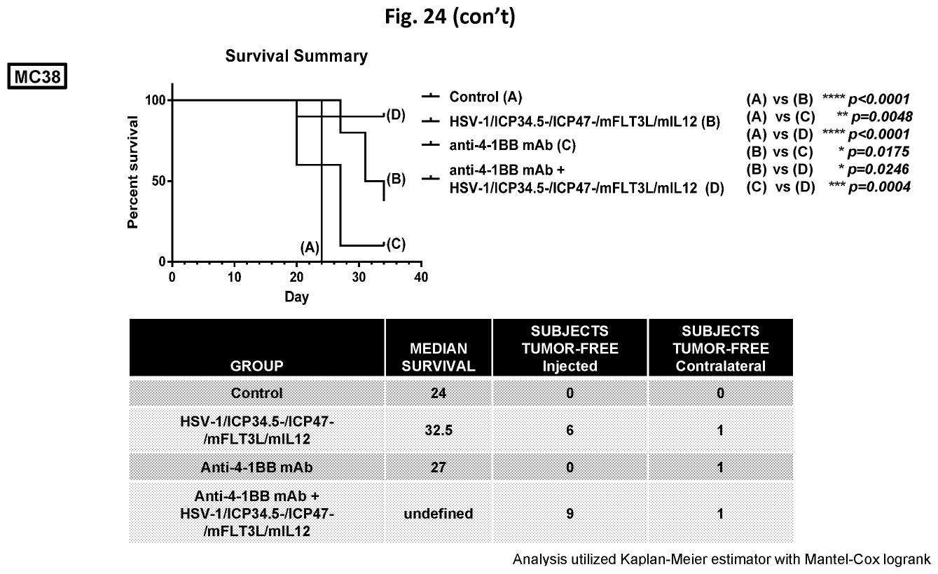

Both single treatments, anti-4-1BB mAb alone, and 5×10⁶ PFU HSV-1/ICP34.5⁻/ICP47⁻/mFLT3L/mIL12 alone, demonstrated significantly increased survival as compared to control treated animals (p=0.0048 and p<0.0001 for each comparison respectively). Survival of 5×10⁶ PFU HSV-1/ICP34.5⁻/ICP47⁻/mFLT3L/mIL12 treated animals was statistically significant as compared to anti-4-1BB mAb alone (p=0.0175). The combination of both treatments, anti-4-1BB mAb plus 5×10⁶ PFU HSV-1/ICP34.5⁻/ICP47⁻/mFLT3L/mIL12, demonstrated significantly increased survival as compared to all other treatment groups (p=0.0246 as compared to 5×10⁶ PFU HSV-1/ICP34.5⁻/ICP47⁻/mFLT3L/mIL12 alone, p=0.0004 as compared to anti-4-1BB mAb alone, and p<0.0001 as compared to control treatment). See FIG. 24.

These data indicate that while either HSV-1/ICP34.5⁻/ICP47⁻/mFLT3L/mIL12 or anti-4-1BB mAb treatment alone led to a significant improvement in overall survival as compared to control treatment, the combination of both treatments resulted in a significantly improved overall survival as compared to either treatment alone.

### Example 15: Study Evaluating Efficacy of HSV-1/ICP34.5⁻/ICP47⁻/mFLT3L/mIL12 in Combination with a Bispecific T Cell Engager (BiTE®) Molecule in a Mouse Colorectal (MC38) Tumor Model

This study evaluates the tolerability and anti-tumor activity of HSV-1/ICP34.5⁻/ICP47⁻/mFLT3L/mIL12 alone or in combination with a bispecific T cell engager (BITE®) molecule in a contralateral mouse MC38 tumor model overexpressing human epithelial cell adhesion molecule (EpCAM).

MC38 tumor cells engineered to express human EpCAM (3×10⁵ cells) are injected subcutaneously in the right and left flanks of female C57BL/6 mice that are engineered to express human CD3 from the endogenous mouse CD3 locus on day 0. Tumor volume (mm³) is measured using electronic calipers twice per week (Q2W). Once tumors reached an average volume of approximately 100 mm³, animals are randomized into groups (10 mice per group) such that the average tumor volume (in both flanks) and the variability of tumor volume at the beginning of treatment administration are uniform across treatment groups. HSV-1/ICP34.5⁻/ICP47⁻/mFLT3L/mIL12 (5×10⁶ PFU/dose) or formulation buffer control is administered intratumorally (on the right side of the animal) every three days for three total injections. The uninjected tumors (contralateral; on the left side of the animal) receive no injection. A BiTE® molecule containing anti-human CD3 and anti-human EpCAM binding domains (150 μg/kg) is administered by intravenous injection once weekly for two total injections. Clinical signs, body weight changes, and survival (mice are removed from study when tumors reached 800 mm³) are measured 2 times weekly until study termination.

1-34. (canceled)

35. An oncolytic herpes simplex virus,
wherein said oncolytic herpes simplex virus:
lacks a functional gene encoding ICP 34.5,
lacks a functional gene encoding ICP 47, and
wherein said oncolytic herpes simplex virus contains:
a nucleic acid sequence encoding Fms-related tyrosine kinase 3 ligand (FLT3L),
a nucleic acid sequence encoding interleukin-12 (IL12), and
a nucleic acid encoding a Kozak sequence;
wherein the nucleic acid sequence encoding FLT3L and the nucleic acid sequence encoding IL12 are both under the control of a cytomegalovirus (CMV) promotor; and
wherein the nucleic acid sequence encoding FLT3L and the nucleic acid sequence encoding IL12 are linked by the polycistronic linker element porcine tescho virus 2a (P2A).

36. The oncolytic herpes simplex virus according to claim 35, wherein said oncolytic herpes simplex virus is a herpes simplex-1 virus (HSV-1).

37. The oncolytic herpes simplex virus according to claim 35, wherein said HSV-1 is modified from strain JS1 deposited at the ECAAC under accession number 01010209.

38. The oncolytic herpes simplex virus according to any one of claims 35-37, wherein said nucleic acid sequence encoding IL12 and said nucleic acid sequence encoding FLT3L are present in the former site of the gene encoding ICP34.5.

39. The oncolytic herpes simplex virus according to claim 38, wherein said oncolytic herpes simplex virus further comprises a bovine growth hormone polyadenylation signal sequence (BGHpA).

40. The oncolytic herpes simplex virus according to claim 39, wherein said nucleic acids encoding CMV, Kozak, Flt3L, P2A, IL12, and BGHpA are present as [CMV]-[Kozak]-[Flt3L]-[P2A]-[IL12]-[BGHpA].

41. The oncolytic herpes simplex virus according to claim 35, wherein said IL12 is present as [P40 subunit]-[GGGGS]-[P35 subunit].

42. The oncolytic herpes simplex virus according to claim 41, wherein the signal peptide in the IL12 P35 subunit is absent.

43. The oncolytic herpes simplex virus according to claim 35, wherein said oncolytic herpes simplex virus comprises:
a FLT3L sequence comprising SEQ ID NO: 1; and
an IL12 sequence comprising SEQ ID NO: 7.

44. The oncolytic herpes simplex virus according to claim 35,
wherein said oncolytic herpes simplex virus is a HSV-1, and
wherein said oncolytic herpes simplex virus comprises:
a CMV promotor comprising SEQ ID NO: 24;
a Kozak sequence comprising SEQ ID NO: 20;
a FLT3L sequence comprising SEQ ID NO: 1;
a P2A sequence comprising SEQ ID NO: 17;
an IL12 sequence comprising SEQ ID NO: 7; and
a BGHpA sequence comprising SEQ ID NO: 21.

45. The oncolytic herpes simplex virus according to claim 40,
wherein said oncolytic herpes simplex virus is a HSV-1, and
wherein said oncolytic herpes simplex virus comprises:
a CMV promotor comprising SEQ ID NO: 24;
a Kozak sequence comprising SEQ ID NO: 20;
a FLT3L sequence comprising SEQ ID NO: 1;
a P2A sequence comprising SEQ ID NO: 17;
an IL12 sequence comprising SEQ ID NO: 7; and
a BGHpA sequence comprising SEQ ID NO: 21.

46. An oncolytic herpes simplex virus, wherein said oncolytic herpes simplex virus is HSV1/ICP34.5⁻/ICP47⁻/FLT3L/IL12, wherein said HSV1/ICP34.5−/ICP47−/FLT3L/IL12 comprises a modified HSV-1 derived from strain JS1, wherein the HSV-1 lacks a functional ICP34.5 encoding gene, lacks a functional ICP47 encoding gene, comprises the following inserted into the former sites of the ICP34.5 gene: [CMV]-[Kozak]-[FLT3L]-[P2A]-[IL12(p40-GGGGS-No SP-p35)]-[BGHpA].

47. A pharmaceutical composition comprising an oncolytic herpes simplex virus according to any one of claims 35, 44, or 46.

48. A kit comprising an oncolytic herpes simplex virus according to any one of claims 35, 44, or 46.


SEQUENCE LISTING (24 sequences)

SEQ ID NO: 1 - DNA, 705 bp, synthetic construct
  misc_feature 1..705: note = huFLT3L
  source 1..705: mol_type = other DNA
atgacagtgctggcgccagcctggagcccaacaacctatctcctcctgctgctgctgctgagctcgggactcagtgggacccaggactgctccttccaacacagccccatctcctccgacttcgctgtcaaaatccgtgagctgtctgactacctgcttcaagattacccagtcaccgtggcctccaacctgcaggacgaggagctctgcggtggcctctggcggctggtcctggcacagcgctggatggagcggctcaagactgtcgctgggtccaagatgcaaggcttgctggagcgcgtgaacacggagatacactttgtcaccaaatgtgcctttcagccacctcccagctgtcttcgcttcgtccagaccaacatctcccgcctcctgcaggagacctccgagcagctggtggcgctgaagccctggatcactcgccagaacttctcccggtgcctggagctgcagtgtcaacctgactcctcaactctgccacctccatggagtccacggcctctggaggcaacagcgccgacagctccgcagccccctctgttactcctactgctgctgcccgtgggcctcctactgctggcagctgcctggtgcctgcactggcagaggacgcggcggaggacaccccgacctggggagcaggtgccccccgtccccagtccccaggacctgctgcttgtggagcac

SEQ ID NO: 2 - protein, 235 aa, synthetic construct
  REGION 1..235: note = huFLT3L
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 3 - DNA, 594 bp, synthetic construct
  misc_feature 1..594: note = huIL12A
  source 1..594: mol_type = other DNA
agaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctag

SEQ ID NO: 4 - protein, 197 aa, synthetic construct
  REGION 1..197: note = huIL12A
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 5 - DNA, 984 bp, synthetic construct
  misc_feature 1..984: note = huIL12B
  source 1..984: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagt

SEQ ID NO: 6 - protein, 328 aa, synthetic construct
  REGION 1..328: note = huIL12B
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 7 - DNA, 1590 bp, synthetic construct
  misc_feature 1..1590: note = human single-chain IL-12 (huIL12B-GGGGS-huIL12A)
  source 1..1590: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggcggtggagggtccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcc

SEQ ID NO: 8 - protein, 530 aa, synthetic construct
  REGION 1..530: note = human single-chain IL-12 (huIL12B-GGGGS-huIL12A)
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 9 - DNA, 696 bp, synthetic construct
  misc_feature 1..696: note = muFl3l
  source 1..696: mol_type = other DNA
atgacagtgctggcgccagcctggagcccaaattcctccctgttgctgctgttgctgctgctgagtccttgcctgcgggggacacctgactgttacttcagccacagtcccatctcctccaacttcaaagtgaagtttagagagttgactgaccacctgcttaaagattacccagtcactgtggccgtcaatcttcaggacgagaagcactgcaaggccttgtggagcctcttcctagcccagcgctggatagagcaactgaagactgtggcagggtctaagatgcaaacgcttctggaggacgtcaacaccgagatacattttgtcacctcatgtaccttccagcccctaccagaatgtctgcgattcgtccagaccaacatctcccacctcctgaaggacacctgcacacagctgcttgctctgaagccctgtatcgggaaggcctgccagaatttctctcggtgcctggaggtgcagtgccagccggactcctccaccctgctgcccccaaggagtcccatagccctagaagccacggagctcccagagcctcggcccaggcagcttttgctcctgctactgctgttgctacctctcacactggtgctgctggcagccgcctggggccttcgctggcaaagggcaagaaggaggggggagctccaccctggggtgcccctcccctcccatccc

SEQ ID NO: 10 - protein, 232 aa, synthetic construct
  REGION 1..232: note = muFl3l
MTVLAPAWSPNSSLLLLLLLLSPCLRGTPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPRPRQLLLLLLLLLPLTLVLLAAAWGLRWQRARRRGELHPGVPLPSHP

SEQ ID NO: 11 - DNA, 579 bp, synthetic construct
  misc_feature 1..579: note = muIl12a
  source 1..579: mol_type = other DNA
agggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcc

SEQ ID NO: 12 - protein, 193 aa, synthetic construct
  REGION 1..193: note = muIl12a
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 13 - DNA, 1005 bp, synthetic construct
  misc_feature 1..1005: note = muIl12b
  source 1..1005: mol_type = other DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcc

SEQ ID NO: 14 - protein, 335 aa, synthetic construct
  REGION 1..335: note = muIl12b
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 15 - DNA, 1599 bp, synthetic construct
  misc_feature 1..1599: note = murine single-chain IL12 (muIl12b-GGGGS-muIl12a)
  source 1..1599: mol_type = other DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggcggtggagggtccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcc

SEQ ID NO: 16 - protein, 533 aa, synthetic construct
  REGION 1..533: note = murine single-chain IL12 (muIl12b-GGGGS-muIl12a)
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 17 - DNA, 66 bp, synthetic construct
  misc_feature 1..66: note = GSG-P2A
  source 1..66: mol_type = other DNA
ggatccggtgccacaaacttctctttgctaaagcaagcaggagatgttgaggaaaaccctgggccc

SEQ ID NO: 18 - protein, 22 aa, synthetic construct
  REGION 1..22: note = GSG-P2A
GSGATNFSLLKQAGDVEENPGP

SEQ ID NO: 19 - DNA, 587 bp, synthetic construct
  misc_feature 1..587: note = IRES sequence
  source 1..587: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacc

SEQ ID NO: 20 - DNA, 10 bp, synthetic construct
  misc_feature 1..10: note = Kozak sequence
  source 1..10: mol_type = other DNA
ctaggccacc

SEQ ID NO: 21 - DNA, 225 bp, synthetic construct
  misc_feature 1..225: note = BGHpA sequence
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 22 - DNA, 36 bp, synthetic construct
  misc_feature 1..36: note = Elastin sequence
  source 1..36: mol_type = other DNA
gttcctggagtaggggtacctggagtgggcggatct

SEQ ID NO: 23 - protein, 12 aa, synthetic construct
  REGION 1..12: note = Elastin sequence
VPGVGVPGVGGS

SEQ ID NO: 24 - DNA, 588 bp, synthetic construct
  misc_feature 1..588: note = CMV sequence
  source 1..588: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc